(19) United States
(12) Patent Application Publication
(10) Pub. No.: US 2008/0187492 A1
(43) Pub. Date: Aug. 7, 2008

(54) Drosophila Models For Diseases Affecting Learning and Memory

(76) Inventors: Sean M.J. McBride, Bronx, NY (US); Thomas A. Jongens, Bala Cynwyd, PA (US); Catherine H. Choi, Ridley Park, NY (US)

Correspondence Address: AMSTER, ROTHSTEIN & EBENSTEIN LLP, 90 PARK AVENUE, NEW YORK, NY, 10016, US

(21) Appl. No.: 11/578,077

(22) PCT Filed: Apr. 14, 2005
(86) PCT No.: PCT/US05/12543

§ 371 Date: Oct. 1, 2007

Related U.S. Application Data
(60) Provisional application No. 60/562,922, filed on Apr. 16, 2004.

(51) Int. Cl. A61K 49/00; A61K 33/14; A61K 31/44; A61K 31/7088; A61P 25/28

(52) U.S. Cl. 424/9.2; 424/677; 514/277; 514/44

(57) ABSTRACT

Methods of evaluating a compound for the ability to reduce a mental defect in a metazoan are provided, where the mental defect is caused by Fragile X syndrome, a tauopathy, Huntington's disease, neurofibromatosis 1, Parkinson's disease. The methods comprise determining whether the compound reduces a mental effect of the analogous disease in a Drosophila melanogaster Also provided are methods of evaluating a compound for the ability to improve learning or memory in a mammal. The methods comprise determining whether the compound improves learning or memory in a Drosophila melanogaster that is deficient in a dFRM1. Additionally, methods of treatment of a mammal deficient in expression of an FMR1 gene are provided. The methods comprise treating the mammal with a compound in a pharmaceutically acceptable excipient, where the compound inhibits expression or activity of a group II or group I metabotropic glutamate receptor (mGluR), an inositol trisphosphate receptor (InsP3R), a glycogen synthase kinase-3β (GSK-3β), or a phosphodiesterase-4 (PDE-4) in the mammal.

### CROSS-REFERENCE TO RELATED APPLICATION

This application claims the benefit of U.S. Provisional Application No. 60/562,922, Filed Apr. 16, 2004.

### BACKGROUND OF THE INVENTION

1. Field of the Invention

The present invention generally relates to models of, and treatments for, diseases affecting learning and memory. More specifically, the present invention describes a Drosophila model for diseases affecting learning and memory and use of that model to identify compounds that are useful in treating the learning and memory-affecting components of those diseases, including Fragile X disease.

2. Description of the Related Art

### REFERENCES CITED

Ackerman S L, Siegel R W. (1986) J Neurogenetics 3, 111-123.
Alarcon J M, Malleret G, Touzani K, Vronskaya S, Ishii S, Kandel E R, Barco A 2004 Neuron 42, 947-59.
Auluck P K, Chan H Y, Trojanowski J Q, Lee V M, Bonini N M. (2002) Science 295, 865-8.
Bakker C E, Oostra B A (2003) Cytogenet Genome Res 100, 111-123.
Bardoni B, Schenck A, Mandel J L. (2001) Brain Res Bull 56, 375-82.
Barth M, Heisenbeg M. (1997) Learn Mem 4, 219-229.
Bastock M, Manning, A. (1955) Behaviour 8, 85-111.
Bastock M (1956) Evolution 10, 421-439.
Berridge M J, Downes C P, Hanley M R. (1989) Cell 59, 411-9.
Berridge M J. (1993) Nature 365, 388-9.
Bourtchouladze R, Lidge R, Catapano R, Stanley J, Gossweiler S, Romashko D, Scott R, Tully T (2003) Proc Natl Acad Sci USA 100, 10518-22.
Bozon B, Kelly A, Josselyn S A, Silva A J, Davis S, Laroche S. (2003) Philos Trans R Soc Lond B Biol Sci. 29, 358 (1432), 805-14.
Brakeman P R, Lanahan A A, O'Brien R, Roche K, Barnes C A, Huganir R L, Worley P F. (1997) Nature 386, 284-8.
Brown V, Jin P, Ceman S, Darnell J C, O'Donnell W T, Tenenbaum S A, Jin X, Feng Y, Wilkinson K D, Keene J D, Darnell R B, Warren S T. (2001) Cell 107, 477-87.
Bullock B P, Habener J F. (1998) Biochemistry 37, 3795-809.
Bushell T J, Jane D E, Tse H W, Watkins J C, Garthwaite J, Collingridge G L (1996) Br J. Pharmacol. 117, 1457-62.
Chen A, Muzzio I A, Malleret G, Bartsch D, Verbitsky M, Pavlidis P, Yonan A L, Vronskaya S, Grody M B, Cepeda I, Gilliam T C, Kandel E R. (2003) Neuron 14; 39(4):655-69.
Cho K, Bashir Z I. (2002) Trends in Neurosciences 25, 405-410.
Cobb M, Ferveur J F. (1996) Behav Proc 35, 35-54.
Comery T A, Harris J B, Willems P J, Oostra B A, Irwin S A, Weiler I J, Greenough W T. (1997) Proc Natl Acad Sci USA 94, 5401-4.
Connolly K, Cook R. (1973) Behaviour 44, 142-165.
Costa R M, Federov N B, Kogan J H, Murphy G G, Stem J, Ohno M, Kucherlapti R, Jacks T, Silva A J. (2002) Nature 415, 526-530.
Darnell J C, Jensen K B, Jin P, Brown V, Warren S T, Darnell R B. (2001) Cell 107, 489-99.
Davis R L. (1993) Neuron 11, 1-14.
Dockendorff T C, Su H S, McBride S M, Yang Z, Choi C H, Siwicki K K, Sehgal A, Jongens T A. (2002) Neuron 34, 973-84.
Dubnau J, Grady L, Kitamoto T, Tully T. (2001) Nature 411, 476-80.
Dudia Y, Jan Y N, Byers D, Quinn W, Benzer, S. (1976) Proc Natl Acad Sci USA 73, 1684-1688.
Duff K, Knight H, Refolo L M, Sanders S, Yu X, Picciano M, Malester B, Hutton M, Adamson J, Goedert M, Burki K, Davies P. (2000) Neurobiol Dis 7, 87-98.
Dujarin F. (1850) Sci Nat Zool 14, 195-206.
Erber J, Masuhr T, Menzel R. (1980) Physiol Entomol 5, 343-358.
Faber T, Joerges J, Menzel R. (1999) Nat Neurosci 2, 74-78.
Feany M B abd Bender W W (2000) Nature 404, 394-398.
Fisch G S, Carpenter N, Holden J J, Howard-Peebles P N, Maddalena A, Borghgraef M, Steyaert J, Fryns. J P. (1999a) Am J Med Genet 83, 308-12.
Fisch G S, Hao H K, Bakker C, Oostra B A. (1999b) Am J Med. Genet. 84, 277-82.
Fisch G S, Simensen R J, Schroer R J, (2002) J. Autism Dev. Disorders 32, 107-114.
Folbergrova J, Haugvicova R, Mares P (2001) Brain Res. 908, 120-9.
Fortini M E et al. (2000) J Cell Biol 150, F23-F29.
Fujii S, Matsumoto M, Igarashi K, Kato H, Mikoshiba K. (2000) Learn Mem. 7, 312-20.
Gailey D A, Jackson F R, Siegel R W. (1982) Genetics 102, 771-782.
Gailey D A, Jackson F R, Siegel R W. (1984) Genetics 106, 613-623.
Godfraind J M, Reyniers E, De Boulle K, D'Hooge R, De Deyn P P, Bakker C E, Oostra B A, Kooy R F, Willems P J. (1996) Am. J. Med. Genet. 64, 246-251.
Goldman-Rakic P. (1995) Neuron 14, 477-485.
Greenspan R J. (1995) Neuron 15, 747-750.
Griffith L C, Verselis L M, Aitken K M, Kyriacou C P, Danho W, Greenspan R J. (1993) Neuron 10, 501-509.
Grimes C A, Jope R S. (2001) J Neurochem. 78, 1219-32.
Guan Z, Giustetto M, Loinvardas S, Kim J H, Miniaci M C, Schwartz J H, Thanos D, Kandel E R (2002) Cell 111, 483-93.
Guo H F, Tong J, Hannan F, Luo L, Zhong Y A. (2000) Nature 403, 895-898.
Hall J C. (1994) Science 264, 1702-1714.
Hagerman R J, Schreiner R A, Kemper M B, Wittenberger M D, Habicht K (1989) Am. J. Med. Gen. 33, 513-518.
Hay D A (1994) Am. J. Med. Gen. 51, 358-363.
Hagerman R J. (1991). Physical and behavioral phenotype, and medical follow up and pharmacology. In Hagerman, R J and A C Silverman (Eds.), Fragile X syndrome: Diagnosis, treatment, and research (pps 282-310). Baltimore, Md.: Johns Hopkins University Press.
Heisenberg M. (1980) In Development and Biology of Drosophila, O, Siddiqi, P. Babu, L. M. Hall and J. C. Hall, eds. (New York: Plenum), pp 373-390.
Heisenberg M. (1994) Central brain function in insects: Genetic studies on the mushroom bodies and central complex in Drosophila, 61-79. Schildenberger, K., and Elsner, N., editors. Neural Basis of Behavioural Adaptations. Gustav, Fischer, and Verlag, New York.
Hinton V J, Brown W T, Wisniewski K, Rudelli R D (1991) Am. J. Med. Genet. 41, 289-294.
Hou J, Kuromi H, Fukasawa Y, Ueno K, Sakai T, Kidokoro Y (2004) J Neurobiol. 60, 249-61.
Huang L, Breakwell N A, Rowan M J, Anwyl R (1997) Eur J Pharmacol. 332, 161-5.
Huang L, Killbride J, Rowan M J, Anwyl R. (1999a) Neuropharmacology. 38, 73-83.
Huang L, Rowan M J, Anwyl R. (1999b) Eur J Pharmacol. 366(2-3), 151-8.
Huber K M, Gallagher S M, Warren S T, Bear M F. (2002) Proc Natl Acad Sci USA. 99, 7746-50.
Hummel T, Krukkert K, Roos J, Davis G, Klambt C. (2000) Neuron 26, 357-70.
Inoue T, Kato K, Kohda K, Mikoshiba K. (1998) J Neurosci. 18, 5366-73.
Jane D E, Pittaway K, Sunter D C, Thomas N K, Watkins J C (1995) Neuropharmacology 34, 851-6.
Joyner M L, Griffith L C. (1997) J Neurosci 17, 9384-9391.
Kane N S, Robichon A, Dickinson, J A, Greenspan, R J. (1997) Neuron 18, 307-314.
Kazemi-Esfarjani, P and Benzer, S (2000) Science 287, 1837-1840.
Khodakhah K, Armstrong C M. (1997) Proc Natl Acad Sci USA 94, 14009-14.
Kreibich T A, Chalasani S H, Raper J A. (2004) J Neurosci. 24, 7085-95.
Kunick, C et al. (2004) Bioorg. Med. Chem. Let. 14, 413-6.
Levin L R, Han P L, Hwang P M, Feinstein P G, Davis R L, Reed R R. (1992) Cell. 68, 479-89.
Li J, Pelletier M R, Perez Valezquez J L, Carlen P L (2002) Mol Cell Neuroscience 19, 138-151.
Mai L, Jope R S, Li X. (2002) J Neurochem. 82, 75-83.
Mak D O., McBride S, and Foskett J K. (1998) Proc Natl Acad Sci USA 95, 15821-5.
Malherbe P, Kratochwil N, Zenner M-T, Piussi J, Diener C, Kratzeisen C, Fischer C, Porter R H P (2003) Molecular Pharmacology 64, 823-832.
McBride S M J, Giuliani G, Choi C, Krause P, Correale D, Watson K, Baker G, Siwicki K K. (1999) Neuron 24, 967-977.
McGuire S E, Le P T, Davis R L. (2001) Science 293, 1330-3.
Morales J, Hiesinger P R, Schroeder A J, Kume K, Verstreken P, Jackson F R, Nelson D L, Hassan B A. (2002) Neuron 34, 961-72.
Nakamura T, Barbara J-G, Nakamura K, Ross W N (1999) Neuron 24, 727-737.
Naples M A, Hampson D R (2001) Neuropharmacology 40, 170-7.
Nimchinsky E A, Oberlander A M, Svoboda K (2001) J. Neuroscience 21, 5139-5146.
Nishiyama M, Hong K, Mikoshiba K, Poo M M, Kato K. (2000) Nature 408, 584-8.
O'Donnell W T, Warren S T. (2002) Annu Rev Neurosci 25, 315-38.
Orgad S, Rosenfeld G, Greenspan R J, Segal D (2000) Genetics 155, 1267-1280.
Ostrerova N, Petrucelli L, Farrer M, Mehta N, Choi P, Hardy J, Wolozin B. (1999) J Neurosci 19, 5782-91.
Otani S, Auclair N, Desce J-M, Roisin M-P, Crepel F (1999) J. Neuroscience 19, 9788-9802.
Otani S, Daniel H, Takita M, Crepel F (2002) J. Neuroscience 22, 3434-3444.
Pagano A, Ruegg D, Litschig S, Stoer N, Stierlin C, Heinrich M, Floersheim P, Prezeau L et al (2000) J Biol Chem 275, 33750-33758.
Paredee W, Melakian H E, Rasmussan D L, Kenneson A, Conn P J, Warren S T. (1999) Neuroscience 94, 185-192.
Parmentier M L, Pin J P, Bockhaert J, Grau Y (1996) J. Neuroscience 16, 6687-6694.
Pascual A, Preat T. (2001) Science 294(5544), 1115-7.
Petralia R S, Wang Y-X, Niedzielski A S, Wenthold, R S (1996) Neuroscience 71, 949-976.
Pommier B, Marie-Claire C, Da Nascimento S, Wang H L, Roques B P, Noble F. (2003) J Neurochem. 85, 454-61.
Power M E. (1943) J Morphol 72, 517-559.
Purpura D P. (1974) Science 186, 1126-1128.
Ramaekers A, Parmentier M-L, Lasnier C, Bockaert J, Grau Y (2001) J. of Comparative Neurology 438, 213-225.
Raymond V, Hamon A, Grau Y, Lapied B (1999) Neuroscience Letters 269, 1-4.
Roman G, Davis R L. (2000) Bioessays 23, 571-81.
Saura C A, Choi S—Y, Beglopoulos V, Malkani S, Zhang D, Rao B S S, Chattarji S, Kelleher R J, Kandel E R, Duff K, Kirkwood A, Shen J (2004) Neuron 42, 23-36.
Schildenberger K. (1984) J Comp Physiol A154, 71-79.
Selkoe D J (2000) Curr Opin Neurobiol. 10, 1837-1840.
Siegel R W, Hall J C. (1979) Prot Natl Acad. Sci. USA 56, 3430-3434.
Son H, Yu I T, Hwang S J, Kim J S, Lee S H, Lee Y S, Kaang B K. (2003) J Neurochem. 85(4):872-81.
Spieth H T. (1974) Annual Review Entomology 19, 383-406.
Stamer K et al. (2002) J Cell Biol 156, 1051-1063.
Steffan et al. (2001) Nature 413, 739-743.
Strausfeld N J. (1976) Atlas of an Insect Brain, p. 214 (Berlin: Springer-Verlag).
Strausfeld N J, Bushbeck E K, Gomez R S. (1995) The arthropod mushroom body: Its functional roles, evolutionary enigmas and mistaken identities, 349-381. Breidback O and W Kutsch, editors. The Nervous Systems of Invertebrates: An Evolutionary and Comparative Approach. Burkhauser Verlag Basel, Switzerland.
Sturtevant A H. (1915) J Anim Behav 5, 351-366.
Takei K, Shin R M, Inoue T, Kato K, Mikoshiba K. (1998) Science 282, 1705-8.
Tamura T, Chiang A S, Ito N, Liu H P, Horiuchi J, Tully T, Saitoe M. Neuron. 2003 Dec. 4; 40(5):1003-11.
Taylor J P, Taye A A, Campbell C, Kazemi-Esfarjani P, Fischbeck K, Min K T (2003) Genes Dev. 17, 1463-8.
Tompkins L, Hall J C, Hall L M. (1980) J Insect Physiology 26, 689-69.
Tompkins L, Gross A C, Hall J C, Gailey D A, Siegal R W. (1982) Behav Genetics 295-307.
Tompkins L, Siegal R W, Gialey D A, Hall J C. (1983) Genetics 13, 565-578.
Tompkins L. (1984) Behav Genetics 14, 411-440.
Tully T, Boynton S, Brandes C, Dura J M, Mihalek R, Preat T, Villela A. (1990) Cold Spring Harbor Symp. Quant. Biol. 55, 203-211.
Tully T, Preat T, Boynton S C, Del Vecchio M. (1994) Cell 79, 35-47.
Tully T, Bourtchouladze R, Scott R, Tallman J (2003) Nat Rev Drug Discov. 2, 267-77.
Van Dam D, D'Hooge R, Hauben E, Reyniers E, Gantois I, Bakker C E, Oostra B A, Kooy R F, De Deyn P P (2000) Behav Brain Res. 117, 127-36.
van Swinderen B and Hall J C (1995) Learning and Memory 2, 49-61.
Villella A, Hall J C. (1996) Genetics 143, 331-344.
Vitolo O V, Sant'Angelo A, Costanzo V, Battaglia F, Arancio O, Shelanski M. (2002) Proc Natl Acad Sci USA. 99, 13217-21.
Wade S M, Lim W K, Lan K L, Chung D A, Nanamori M, Neubig R R. (1999) Mol. Pharmacol. 56, 1005-13.
Wan L, Dockendorff T C, Jongens T A, Dreyfuss G. (2000) Mol Cell Biol. 22, 8536-47.
Wang H, Storm D R (2003) Mol. Pharmacol. 63, 463-8.
Weiler I J, Greenough W T (1999) Am. J. Med. Genetics 83, 248-252.
Williams R S, Cheng L, Mudge A W, Harwood A J. (2002) Nature 417, 292-5.
Wittmann C W et al. (2001) Science 293, 711-714.
Wright-Talamante C, Cheema A, Riddle J E, Luckey D W, Taylor A K, Hagerman R J (1996) Am. J. Med. Gen. 64, 350-355.
Yang M Y, Armstrong J D, Vilinsky I, Strausfeld N J, Kaiser K. (1995) Neuron 1, 45-54.
Yin J C P, Wallach J S, Del Vecchio M, Wilder E L, Zhou H, Quinn W G, Tully T. (1994) Cell 79, 49-58.
Yin J C P, Del Vecchio M, Zhou H, Tully T. (1995) Cell 81, 107-115.
Yuan J P, Kiselyov K, Shin D M, Chen J, Shcheynikov N, Kang S H, Dehoff M H, Schwarz M K, Seeburg P H, Muallem S, Worley P F (2003) Cell 114, 777-789.
Zars T, Fischer M, Schulz R, Heisenberg M. (2000) Science 288, 672-5.
Zeng W, Mak D-O D, Li Q, Shin D M, Foskett J K, Muallem S (2003) Current Biology 13, 872-876.
Zhang H L, Eom T, Oleynikov Y, Shenoy S M, Liebelt D A, Dictenberg J B, Singer R H, Bassell G J. (2001b) Neuron 24, 261-75.
Zhang Y Q, Bailey A M, Matthies H J, Renden R B, Smith M A, Speese S D, Rubin G M, Broadie K. (2001a) Cell 107, 591-603.

Learning and memory (cognitive abilities) can be examined in Drosophila by utilizing several available learning and memory paradigms. The most popular is a classical conditioning paradigm in which the flies learn to associate electric shocks with olfactory cues (Dudia et al, 1976; Davis, 1993; Tully, 1994). An alternative paradigm to study learning and memory in Drosophila is called conditioned courtship, wherein a male fly learns to modify his courtship behavior after experience with an unreceptive female; it is a multi-sensory paradigm involving associations from more then one sensory input (Siegel and Hall, 1979; for review see Hall, 1994). It is a more complex associative learning paradigm and was utilized to assay learning and memory in this article to elucidate the role of dFMR1 in learning and memory (Tompkins et al, 1980; Tompkins et al, 1982; Tompkins et al, 1983; Tompkins et al, 1984; Ackerman and Siegel, 1986). Courting male flies perform a characteristic sequence of behaviors: orienting toward and following the female, tapping her with his forelegs, vibrating one or both wings, licking her genitalia, and attempting copulation (Sturtevant, 1915; Bastock and Manning, 1955; Bastock, 1956). These behaviors are repeated with some variation until successful copulation occurs. Virgin females will generally respond by mating; however, recently mated females will be unreceptive and will not allow copulation to occur (Spieth, 1974), they display different behaviors (Bastock and Manning, 1955; Connolly and Cook, 1973) and have an altered, although somewhat overlapping, pheromonal profile (Cobb and Ferveur, 1996). The naive male will find a previously mated female to have a pheromonal repertoire that is less provocative then that of a virgin female target. A naive male paired with a mated female will initially court her, but his courtship activity soon decreases; after 1 hour of experience with the mated female, his courtship when subsequently paired with a virgin female remains depressed for 2-3 hours (Siegel and Hall, 1979). This effect is not a general suppression of all courtship activity since a male's tendency to court an immature male is not suppressed (Gailey et al, 1984). Also, experience with a virgin female does not depress courtship towards a subsequent virgin female (Gailey et al, 1982; Gailey et al, 1984). These behaviors are quantified as a courtship index (CI) which is defined as the percentage of time a male fly spends performing any of the six courtship steps toward a target female in a ten minute test period. A decrease in CI after training with a previously mated female is indicative of behavioral plasticity in the form of learning or memory.

In Drosophila there are five phases of memory as have been dissected out by several genetic and pharmacological studies (Greenspan, 1995). Depending on when the fly is assayed there is an immediate recall at 0-2 minutes post training; short-term memory out to 1 hour; medium-term memory out to 6 hours; anesthesia resistant memory out to two days; and long-term memory which lasts up to 9 days post training and appears to be protein synthesis dependent (Tully et al, 1990; Tully et al, 1994; Yin et al, 1994; Yin et al, 1995; Greenspan, 1995). Li addition, in the conditioned courtship paradigm, learning during training can be assayed by comparing the decrease in CI during the first ten minutes after the male is paired with an unreceptive female with the CI of the last ten-minute period of the pairing. Wild type flies typically show a 40% or more decrease in courtship activity (Joyner and Griffith, 1997; Kane et al, 1997).

Disorders affecting learning and memory. Several human diseases that have a significant effect on learning and memory have analogous models in Drosophila. These include Fragile X syndrome, various tauopathies including Alzheimer's disease, Alzheimer's disease resulting from alterations in presenilin or amyloid precursor protein, Huntington's disease, other polyglutamine diseases, neurofibromatosis 1, and Parkinson's disease. See, also, Foltini et al., 2000.

Fragile X is typically caused by an expansion in the number of tri-nucleotide repeats (CGG) within the FMR1 gene resulting in silencing of transcription by hyper-methylation, but has also been found in rare cases to be the result of deletions of the FMR1 gene. FMR1 is an RNA binding protein that is highly expressed in neurons of the central nervous system and thought to have a role in synaptogenesis and axonal arborization. FMR1 has also been implicated in the regulation of mRNA expression and trafficking at the synapse (Zhang et al, 2001b). Pleiotropic effects of this gene are not unexpected considering studies estimate that it interacts with 4% of mRNAs in humans and it is expressed in all stages of development in a ubiquitous fashion (Brown et al, 2001, Darnell et al, 2001 and O'Donnell and Warren, 2002). Fragile X syndrome in humans affects 1 in 4,000 males and 1 in 8,000 females and is associated with clinically relevant behaviors that include sleep disorders, attention deficit disorder, hyperactivity, and autistic behavior (Hagerman, 1991, Fisch et al, 1999; Bardoni et al, 2001; O'Donnell and Warren, 2002). Associated physical abnormalities include maxillofacial structure, macroorchidism in male patients, abnormalities in dendritic spine morphology and hyper-extensible joints (O'Donnell and Warren, 2002). The most prominent clinical feature of Fragile X syndrome, however, is mental retardation ranging from mild to severe with progressive cognitive decline (Hagerman et al, 1989; Hay, 1994; Wright-Talamante et al, 1996; Fisch et al, 2002). One proposed explanation of the learning and memory deficits is altered shape and number of dendritic spines. This phenomenon was observed in FMR1 knockout mice at 16 weeks of age (Comery et al, 1997), although a later study found alterations in density and length only within the first 4 weeks of postnatal development (Nimchinsky et al, 2001). The phenotype of abnormal dendritic spine morphology has been identified in affected humans at autopsy (Hinton et al, 1991) and is consistent with the theory that dendritic spine dysgenesis may be involved in mental retardation in humans (Purpura, 1974).

In mammals, experiments altering the expression level of FMR1 are complicated by the fact that there are two related genes, namely Fragile X related proteins (FXRP) 1 and 2, which are suspected to compensate for phenotypic deficits in the knockout mouse model (Bakker and Oostra, 2003). The mouse knockout model does recapitulate several of the aspects of the disease including macroorchidism in males, abnormal dendritic morphology at specific time points and an enhanced response to acoustic startle (Bakker and Oostra, 2003). The learning and memory deficits in the knockout mice are very subtle and have often been difficult to replicate (Paradee et al, 1999, Fisch et al, 1999a, Fisch et al, 1999b, Van Dam et al, 2000 and Bakker and Oostra, 2003). In Drosophila there is only one gene, dFMR1, which shares extensive amino acid homology and conservation of several key domains including KH domains, RGG box and the ribosomal association domain (Wan et al, 2000). Three previous studies all used homozygous knockout dFMR1 lines to attempt to model the human disease in Drosophila. The findings of these studies included altered circadian rhythms, altered synaptic arborization, altered activity at the neuromuscular junction (which was partially rescued by altering levels of the MAP1B homologue, futsch) and altered courtship levels (Dockendorff et al, 2002; Morales et al, 2002; Zhang et al, 2001; Hummel et al, 2000). However, the overriding clinical feature of Fragile X syndrome is cognitive deficits.

Tauopathies are diseases implicating the microtubule-binding protein tau. Tau stabilizes microtubules, which is important for the production and maintenance of neurites. It is believed that in Alzheimer's disease, abnormally phosphorylated and aggregated forms of tau accumulate in neurofibrillary tangles, which are thought to inhibit transport of amyloid precursor protein (APP) into axons and dendrites, causing its accumulation in the cell body (Stamer et al., 2002). A transgenic Drosophila expressing mutant human tau mimics the Alzheimer's disease (Wittmann et al., 2001). Another Drosophila model for Alzheimer's are those having mutations in the presenilin 1 gene, which is involved in cleavage of the β-amyloid precursor protein (βAPP) (reviewed in Selkoe, 2000). As used herein, Alzheimer's disease is defined as a tauopathy, even in cases where tau may not be involved in the pathology.

There are also Drosophila models for Huntington's disease (Kazemi-Esfarjani and Benzer, 2000; Steffan et al., 2001), neurofibromatosis 1 (Guo et al, 2000) and Parkinson's disease (Feany and Bender, 2000; Auluck et al., 2002).

Based on the above discussion, there is a need for further development of Drosophila models for human diseases causing mental defects. The present invention addresses that need.

### SUMMARY OF THE INVENTION

Accordingly, the inventors have discovered that certain characteristics of Drosophila courtship are useful for separating and monitoring components of learning and memory, particularly as models of human diseases affecting learning and memory.

Thus, in some embodiments, the invention is directed to methods of evaluating a compound for the ability to reduce a mental defect in a metazoan. In these embodiments, the mental defect is caused by a disease, where the disease is Fragile X syndrome, a tauopathy (including Alzheimer's disease), Huntington's disease, neurofibromatosis 1, Parkinson's disease, and a disease analogous in the metazoan to Fragile X syndrome, a tauopathy, Huntington's disease, neurofibromatosis 1, or Parkinson's disease. The methods comprise determining whether the compound reduces a mental effect of the analogous disease in a Drosophila melanogaster.

In other embodiments, the invention is directed to methods of evaluating a compound for the ability to improve learning or memory in a mammal. The methods comprise determining whether the compound improves learning or memory in a Drosophila melanogaster that is deficient in a dFMR1 or with altered function of at least one presenilin gene.

The inventors have also discovered that inhibitors of expression or activity of group II or group III metabotropic glutamate receptors (mGluR), inositol trisphosphate receptors (InsP3R)(including lithium compounds such as LiCl), glycogen synthase kinase-3β (GSK-3β), or phosphodiesterase-4 (PDE-4) are useful for reversing the mental deficiencies caused by diseases affecting learning or memory.

Thus, the invention is also directed to methods of improving learning or memory in a mammal. The methods comprise treating the mammal with a compound in an amount sufficient to improve learning or memory in the mammal, where the compound inhibits expression or activity of a group II or group III metabotropic glutamate receptor (mGluR), an inositol trisphosphate receptor (InsP3R), a glycogen synthase kinase-3β (GSK-3β), or a phosphodiesterase-4 (PDE-4) in the mammal.

In additional embodiments, the invention is directed to methods of treating a mammal having Fragile X disease or a non-human disease analogous to Fragile X disease. The methods comprise treating the mammal with a compound that inhibits expression or activity of a group II or group III mGluR, an InsP3R, a GSK-3β, or a PDE-4 in the mammal.

Additionally, the invention is directed to methods of treating a mammal with Alzheimer's disease or a non-human disease analogous to Alzheimer's. The methods comprise treating the mammal with a compound that inhibits expression or activity of a group II or group III mGluR, an InsP3R, a GSK-3β, or a PDE-4 in the mammal.

The invention is also directed to kits for treating a mammal deficient in expression of an FMR1 gene, or having Alzheimer's disease or a non-human disease analogous to Alzheimer's disease. The kits comprise (a) a compound in a pharmaceutically acceptable excipient, where the compound inhibits expression or activity of a group II or group III mGluR, an InsP3R, a GSK-3β, or a PDE-4, and (b) instructions directing the use of the compound for treating the mammal.

In further embodiments, the invention is directed to the use of a compound for the manufacture of a medicament for the treatment of a mammal having Fragile X disease, Alzheimer's disease, neurofibromatosis 1, or a non-human disease analogous to Fragile X disease, Alzheimer's disease or neurofibromatosis 1. In these embodiments, the compound inhibits expression or activity of a group II or group III mGluR, an InsP3R, a GSK-3β, or a PDE-4.

The invention is additionally directed to the use of a compound that inhibits expression or activity of a group II or group III mGluR, an InsP3R, a GSK-3β, or a PDE-4 in the treatment of a mammal having Fragile X disease, neurofibromatosis 1, Alzheimer's disease or a non-human disease analogous to Fragile X disease, neurofibromatosis 1, or Alzheimer's disease.

### BRIEF DESCRIPTION OF THE DRAWINGS

For FIGS. 1-5, *=p<0.005, **=p<0.0005, ***=p<0.0001. All males are 5 days old at testing for courtship behaviors (and are placed with fresh food the night before courtship assays), and 6-7 days old for testing in locomotor, olfactory and visual assays. All virgin female targets are 4 days old, all females used as previously mated training targets are five days old and observed to have mated the night before testing.

w1118: the background genotype
dfmr1-3: homozygous dFMR1
Rescue: dfmr1-3+wild type rescue fragment
FS: dfmr1-3+frame shifted rescue fragment
n's are 18 to 41 for all groups.

FIG. 1 is graphs of experimental results showing the effect of dFMR1 expression on learning during training and on immediate recall. Mean CIs (±SEM) are plotted, Ns are indicated above each bar for all groups. Black bars, w1118; open bars, dFMR1-3; blue bars, Rescue (dFMR1-3+wild type rescue fragment); hatched bars, FS (dFMR1-3+frame shifted rescue fragment). Panel A shows results when the male flies are placed in a training chamber with a previously mated female for one hour. The amount of time the male spends courting (CIs) in the first ten-minute interval is compared to the amount of time the male spends courting the female target in the last ten-minute interval (CIs). The initial and final courtship levels of w1118 and dFMR1-3 are similar to each other and show significant depression from the initial to final CIs. The initial and final courtship levels of Rescue and FS are similar to each other and show significant depression from the initial to final intervals. Panel B shows results when, after one training session with a previously mated female, the male is placed with a virgin target female for a ten-minute interval. This was then compared to the courtship of naive males placed in the training chamber for one hour with no female, and then placed with a virgin target female for a ten-minute interval. The w1118 and Rescue lines show depression of courtship activity after training compared to naive trained males. dFMR1-3 and FS lines display no depression relative to naive trained males.

FIG. 2 is graphs of experimental results showing the effect of MPEP (1,000 μg/ml) on naive courtship behavior and on the quality of naive courtship behavior. Mean CIs (±SEM) are plotted, Ns are indicated above each bar for all groups. The food was either control (CT) or the same control food with the addition of MPEP (M). The position of the CT or M are indicative of the point at which the group was on the particular food. The first letter indicates the food type that the larvae grew up on, and the second letter indicates the food type that the adult fly was placed on within four hours of eclosion. Black bars, CT-CT Rescue (dFMR1-3+wild type rescue fragment); hatched bars, CT-M Rescue; blue bars, CT-CT FS (dFMR1-3+frame shifted rescue fragment); open bars, CT-M FS. Panel A shows the results when naive males were placed in the training chamber for one hour with no female, and then placed with a virgin target female for a ten-minute interval. The naive courtship behavior was reduced by high MPEP concentration in Rescue and FS lines, and it was depressed to similar levels by high (433 μM) MPEP treatment in the Rescue and FS groups. Panel B shows the results when naïve males were placed in the training chamber for one hour with no female, and were then placed with a virgin target female for a ten-minute interval. The CT-CT FS group along with the two high MPEP groups failed to progress to later steps in courtship behavior relative to the CT-CT Rescue group.

FIG. 3 is graphs of experimental results showing the effect of MPEP (200 μg/ml or 20 μg/ml), lithium chloride (LiCl) (5 or 50 mM), LY341495 (400 nM), or NaCl (5 or 50 mM), on naive courtship behavior, locomotion, visual acuity, and olfaction. Mean CIs (±SEM) are plotted, Ns are indicated above each bar for all groups. The food was either control (CT) or the control food with the addition of MPEP (M). The position of the CT or M are indicative of the point at which the group was on the particular food. The first letter indicates the food type that the larvae grew up on, and the second letter indicates the food type that the adult fly was placed on within four hours of eclosion. Black bars, CT-CT Rescue (dFMR1-3+wild type rescue fragment); hatched bars, CT-M Rescue; blue bars, CT-CT FS (dFMR1-3+frame shifted rescue fragment); open bars, CT-M FS; gray bars, M-M Rescue; green bars, M-CT Rescue; yellow bars, M-M FS; red, M-CT FS. Panel A shows the results when naive males were placed in the training chamber for one hour with no female, and then placed with a virgin target female for a ten-minute interval. When Rescue flies were raised on CT food and then placed on M food as adults, courtship activity was depressed relative to CT-CT Rescue flies. When FS flies were raised on CT food and then placed on M food as adults, there was a significant increase in courtship activity relative to CT-CT FS flies. Rescue and FS flies on M food in development and then placed on either M of CT food courted as vigorously of CT-CT Rescues flies. M-M FS and M-CT FS groups showed courtship levels similar to CT-CT Rescue flies. Therefore, MPEP in development can rescue the naive courtship phenotype regardless of whether or not the flies receive it as adults. Panel B shows the results when naive males were placed in the training chamber for one hour with no female, and then placed with a virgin target female for a ten-minute interval. Even though the CT-M rescue flies showed low amount of time involved in courtship as naive flies, they still progressed to later phases of courtship to similar levels of all other groups excepting the CT-CT FS group. A higher percentage of the CT-CT FS group failed to advance to later stages of courtship compared to all other groups. Panel C shows the results with a locomotor assay. In that assay, flies of each genotype (n=18-22) were placed in the chambers where courtship is assayed with a line drawn down the center of the covering microscope slide. Every time a fly crossed the line in a two-minute period was then scored (Griffith et al, 1993). Flies of all genotypes had similar locomotor activity profiles. Panel D shows the results with an olfactory assay. In that assay, an olfactory trap was designed and flies were loaded into it in 4 groups of 10 per genotype. The number of flies that were caught in the trap at 36 and 60 hours afterwards was then scored (Orgad et al, 2000). No significant differences were found between the groups. Panel E shows the results with a visual assay. In that assay, flies of each genotype (four groups of twenty flies) were loaded into a Y maze that is totally covered in foil (in total darkness) except for the last inch of one branch of the Y maze (Orgad et al, 2000). Flies were given 2 minutes, then the number of the flies that have entered the chamber having the light shown into it are scored. There was no apparent difference in the ability of the flies to detect light. Panel F shows the results of the treatments with LY341495, LiCl at concentrations of 5 mM and 50 mM, NaCl at concentrations of 5 mM and 50 mM and MPEP (at 20 μg/ml) on FS naive courtship. LY341495, LiCl at both concentrations of 5 mM and 50 mM, and MPEP (at 20 μg/ml) restored naive courtship level, whereas NaCl had no effect on FS flies. Panel G shows that LiCl (both concentrations), MPEP (20 μg/ml), LY341495 (400 nM), and NaCl at 50 mM suppress naive courtship in a test for naive courtship levels in Rescue flies, with 5 mM NaCl having no effect.

FIG. 4 is graphs of experimental results showing the effect MPEP (200 μg/ml), LiCl (5 and 50 mM), MPEP 20 μg/ml, LY341495 (400 nM) NaCl (5 and 50 mM) on learning during training, immediate recall, short-term memory and discrimination. Mean CIs (+/−SEM) are plotted, Ns are indicated above each bar for all groups. The food was either control (CT) or the same control food with the addition of MPEP (M). The position of the CT or M are indicative of the point at which the group was on the particular food. The first letter indicates the food type that the larvae grew up on, and the second letter indicates the food type that the adult fly was placed on within four hours of eclosion. Black bars, CT-CT Rescue (dFMR1-3+wild type rescue fragment); hatched bars, CT-M Rescue; blue bars, CT-CT FS (dFMR1-3+frame shifted rescue fragment); open bars, CT-M FS; gray bars, M-M Rescue; green bars, M-CT Rescue; yellow bars, M-M FS; red, M-CT FS. Panel A shows the results when the male flies are placed in a training chamber with a previously mated female for one hour. The amount of time the male spends courting in the first ten-minute interval is compared to the amount of time the male spends courting the female target in the last ten-minute interval. The initial and final courtship levels of all groups show significant depression from the initial to final intervals indicating that all groups demonstrated learning during training. This demonstrated that treatment by MPEP in development or adulthood does not abolish learning during training. Panel B shows the results when, after one training session with a previously mated female, the male is placed with a virgin target female for a ten-minute interval. This was then compared to the courtship of naive males placed in the training chamber for one hour with no female, and then placed with a virgin target female for a ten-minute interval. The CT-M Rescue line shows depressed courtship activity immediately after training. CT-CT FS flies court just as vigorously immediately after training as naive CT-CT FS flies. All Rescue groups demonstrate depression of courtship activity immediately after training relative to group matched naive flies. The remaining FS groups that were treated with MPEP, all display experience dependent reduction of courtship activity immediately after training when compared to group matched naives. Panel C shows the results when, after a one hour training session with a previously mated female, the female is removed and the male is placed in a holding chamber for 60 minutes, then subsequently placed in a testing chamber with a virgin female target to assess short-term memory. The CT-M Rescue line showed depressed courtship activity at 60 minutes after training. The CT-CT FS flies courted just as vigorously at 60 minutes after training as naive CT-CT FS flies. The Rescue groups treated with MPEP in development, adulthood or in both development and adulthood demonstrated depression of courtship activity at 60 minutes after training relative to group matched naive flies. The remaining FS groups that were treated with MPEP in development alone, adulthood alone, or in both development and adulthood display experience dependent reduction of courtship activity at 60 minutes after training when compared to group matched naives. Panel D shows whether there is a difference in the amount of time a naive male spends courting a virgin female compared to a previously mated female. In these experiments, only the CT-M Rescue and CT-CT Frame shift lines did not spend significantly more time courting virgin female targets. Panel E shows that LY341495, both concentrations of LiCl, and MPEP restored short-term memory in FS flies, whereas NaCl had no effect. Panel F shows the results of the treatments on the short term memory of Rescue flies, with no effect of treatment by either concentration of NaCl, LY341495 or low MPEP. However, both concentrations of LiCl disrupted short term memory in Rescue flies.

FIG. 5 shows binding sequences relevant to the present invention. Panel A shows the putative MPEP binding pocket of mGluR5 (Malherbe et al, 2003 and Pagano et al, 2000) compared to the aligned Drosophila mGluR sequences, critical amino acids in bold. Panel B shows the putative Gi activity/binding motif, with critical amino acids in bold. Panel C shows the putative Gq binding motif, where the relative amino acid spacing is numbered and critical amino acids are in bold. Panel D shows the homology of the Drosophila mGluRs compared to Human mGluRs.

FIG. 6 shows a diagram of a proposed mechanism of action of MPEP on signal transduction.

FIG. 7 is graphs of experimental results showing the effect of 200 μg/ml MPEP on naive courtship behavior, locomotion, visual acuity, and olfaction in 20-day-old flies. Mean CIs (±SEM) are plotted, Ns are indicated above each bar for all groups. The levels of significance are indicated (*=p<0.05, **=p<0.005,***=p<0.0001). The food was either control (CT) or the control food with the addition of MPEP (M). The position of the CT or M are indicative of the point at which the group was on the particular food. The first letter indicates the food type that the larvae grew up on, and the second letter indicates the food type that the adults fly was placed on within four hours of eclosion. Black bars, CT-CT Rescue (dFMR1-3+wild type rescue fragment); hatched bars, CT-M Rescue; blue bars, CT-CT FS (dFMR1-3+frame shifted rescue fragment); open bars, CT-M FS; gray bars, M-M Rescue; green bars, M-CT Rescue; yellow bars, M-M FS; red, M-CT FS. Panel A shows the results when naive males were placed in the training chamber for one hour with no female, and then placed with a virgin target female for a ten-minute interval. When Rescue flies were raised on CT food and then placed on M food as adults, courtship activity was depressed relative to CT-CT Rescue flies. When FS flies were raised on CT food and then placed on M food as adults, there is a significant increase in courtship activity relative to CT-CT FS flies. Rescue flies placed on M food in development and then placed on either M or CT food courted as vigorously of CT-CT Rescue flies. The M-M FS, but not M-CT FS, groups showed courtship levels similar to CT-CT Rescue flies. Therefore, MPEP in adulthood can rescue the naive courtship phenotype of FS flies regardless of whether or not the flies receive MPEP in development. Panel B shows results when naive males were placed in the training chamber for one hour with no female, and then placed with a virgin target female for a ten-minute interval. Even though the CT-M rescue flies showed low amount of time involved in courtship as naive flies, they still progressed to later phases of courtship to similar levels of all other groups excepting the CT-CT FS and M-CT FS groups. A higher percentage of the CT-CT FS and M-CT FS groups failed to advance to later stages of courtship compared to all other groups. Panel C shows the results with a locomotor assay. In this assay, each genotype of flies was placed in the chambers where courtship is assayed with a line drawn down the center of the covering microscope slide. Every time a fly crossed the line in a two-minute period was then scored. Flies of all genotypes had similar locomotor activity profiles. Panel D shows the results with an olfactory assay. In this assay, an olfactory trap was designed (containing yeast as an attractant) and flies were loaded into it in 4 groups of 10 per genotype. The number of flies that were caught in the trap at 24 and 60 hours afterwards was then scored. No significant differences were found between the groups. Panel E shows the results with a visual assay. For each genotype, four groups of twenty flies were loaded into a Y maze that is totally covered in foil (in total darkness) except for the last inch of one branch of the Y maze. Flies were given 2 minutes, then the number of the flies that have entered the chamber having the light shown into it were scored. There was no difference in the ability of the flies to detect light.

FIG. 8 is a graph of experimental results showing the effect of 200 μg/ml MPEP on learning during training in 20-day-old flies. Mean CIs (+/−SEM) are plotted, Ns are indicated above each bar for all groups. The levels of significance are indicated ((*=p<0.05, **=p<0.005,***=p<0.0001). The food was either control (CT) or exactly the same control food with the addition of MPEP (M). The position of the CT or M are indicative of the point at which the group was on the particular food. The first letter indicates the food type that the larvae grew up on, and the second letter indicates the food type that the adults fly was placed on within four hours of eclosion. Black bars, CT-CT Rescue (dFMR1-3+wild type rescue fragment); hatched bars, CT-M Rescue; blue bars, CT-CT FS (dFMR1-3+frame shifted rescue fragment); open bars, CT-M FS; gray bars, M-M Rescue; green bars, M-CT Rescue; yellow bars, M-M FS; red, M-CT FS. The male flies were placed in a training chamber with a previously mated female for one hour. The amount of time the male spends courting in the first ten-minute interval was compared to the amount of time the male spends courting the female target in the last ten-minute interval. There were no differences between the courtship activity in the two intervals in the CT-CT FS group, indicating that no leaning during training occurred. The initial and final courtship levels of all other groups showed significant depression from the initial to final intervals indicating that all other groups demonstrated learning during training. This demonstrates that treatment of FS flies by MPEP in development, adulthood or both is sufficient to restore learning during training in FS flies.

FIG. 9 is a graph of experimental results showing the effect of 200 μg/ml MPEP on immediate recall, short-term memory and discrimination in 20-day-old flies. Mean CIs (±SEM) are plotted, Ns are indicated above each bar for all groups. The levels of significance are indicated (*=p<0.05, **=p<0.005,***=p<0.0001). The food was either control (CT) or exactly the same control food with the addition of MPEP (M). The position of the CT or M are indicative of the point at which the group was on the particular food. The first letter indicates the food type that the larvae grew up on, and the second letter indicates the food type that the adults fly was placed on within four hours of eclosion. Black bars, CT-CT Rescue (dFMR1-3+wild type rescue fragment); hatched bars, CT-M Rescue; blue bars, CT-CT FS (dFMR1-3+frame shifted rescue fragment); open bars, CT-M FS; gray bars, M-M Rescue; green bars, M-CT Rescue; yellow bars, M-M FS; red, M-CT FS. Panel A shows the results during a training session with a previously mated female, where the male was placed with a virgin target female for a ten-minute interval. This was then compared to the courtship of naive males placed in the training chamber for one hour with no female, and then placed with a virgin target female for a ten-minute interval. The CT-M Rescue line showed depressed courtship activity immediately after training. CT-CT FS flies court just as vigorously immediately after training as naive CT-CT FS flies. All Rescue groups demonstrate depression of courtship activity immediately after training relative to group matched naive flies. The remaining FS groups that were treated with MPEP, all display experience dependent reduction of courtship activity immediately after training when compared to group matched naives. Panel B shows the results after a one hour training session with a previously mated female, when the female was removed and the male placed in a holding chamber for 60 minutes, then subsequently placed in a testing chamber with a virgin female target to assess short-term memory. The CT-M Rescue line showed depressed courtship activity at 60 minutes after training. The CT-CT FS flies courted just as vigorously at 60 minutes after training as naive CT-CT FS flies. The Rescue groups treated with MPEP in development, adulthood or in both development and adulthood demonstrate depression of courtship activity at 60 minutes after training relative to group matched naive flies. The remaining FS groups that were treated with MPEP in development alone, adulthood alone, or in both development and adulthood displayed experience-dependent reduction of courtship activity at 60 minutes after training when compared to group-matched naives. Panel C shows whether there is a difference in the amount of time a naive male spends courting a virgin female compared to a previously mated female. Only the M-M Rescue and M-M FS lines spent significantly more time courting virgin female targets then previously mated targets.

FIG. 10 is a graph showing the effect of MPEP on learning during training in a Drosophila model of Alzheimer's disease expressing reduced amounts of presenilin. All flies have reduced presenilin level. Black bars are 5 day old flies raised on kept on control food (N=18) which display learning during training (p<0.05). Hatched bars represent 30 day old flies kept on control food (N=36) which do not show learning during training. Blue bars represent 30 day old flies that were moved to food supplemented with 200 μM MPEP on day 5, and moved back to control food the day before testing (N=20), which do show learning during training. It is evident from this data that, in flies with reduced presenilin levels, there is an age dependent impairment in learning during training. This impairment is prevented by treatment with MPEP.

FIG. 11 is a graph showing the effect on courtship index of MPPG and MTPG on 5 day old adult FS flies.

FIG. 12 is a graph showing the effect on courtship index of MPPG and MTPG on 5 day old adult Rescue flies.

FIG. 13 is a graph showing the effect on courtship index of 5 day old FS and Rescue flies without pharmacologic treatment.

FIG. 14 is a graph showing the effect on courtship index of FS and Rescue flies treated with MPEP on 5 day old flies.

FIG. 15 is a graph showing the effect on courtship index of FS and Rescue flies treated with LY34145 on 5 day old flies.

FIG. 16 is a graph showing the effect on courtship index of FS and Rescue flies treated with MPPG on 5 day old flies.

FIG. 17 is a graph showing the effect on courtship index of FS and Rescue flies treated with MTPG on 5 day old flies.

FIG. 18 is micrographs and graphs showing the relationship between drug treatment and penetrance of the fusion of MB β-lobes.

FIG. 19 is an illustration of relevant signal transduction pathways.

### DETAILED DESCRIPTION OF THE INVENTION

The present invention is based in part on the discovery that certain characteristics of Drosophila courtship are useful for separating and assessing components of learning and memory, particularly as models of human diseases affecting learning and memory. This discovery has led to the development of assays for evaluating compounds for the ability to reduce mental defects.

Thus, in some embodiments, the invention is directed to methods of evaluating a compound for the ability to reduce a mental defect in a metazoan. The methods comprise determining whether the compound reduces a mental effect of an analogous disease in a Drosophila, preferably D. melanogaster. In preferred embodiments, the mental defect is caused by a disease, where the disease is Fragile X syndrome, a tauopathy such as Alzheimer's disease, Huntington's disease, neurofibromatosis 1, Parkinson's disease or a disease analogous in the metazoan to Fragile X syndrome, a tauopathy, Huntington's disease, neurofibromatosis 1, or Parkinson's disease. As previously discussed, there are Drosophila models for Fragile X syndrome, a tauopathy such as Alzheimer's disease, Huntington's disease, neurofibromatosis 1, and Parkinson's disease.

In preferred embodiments, the mental defect is in memory, orientation, learning, attention, reasoning, language, and/or the ability to perform simple tasks. These defects can be measured by any method known in the art for the metazoan in question. For example, in humans, the Alzheimer's Disease Assessment Scale can reliably determine the extent and nature of the mental defect.

These methods are useful for evaluating a compound for reducing mental defects for any disease causing mental defects for which there is an analogous disease in a Drosophila melanogaster. In some preferred embodiments, the disease is Fragile X syndrome and the analogous disease in a Drosophila is caused by a deficiency in a dFMR1 protein. See, e.g., Examples 1 and 2.

In other preferred embodiments, the disease is a tauopathy and the analogous disease in a Drosophila is caused by expression of a human tau protein, preferably a mutant human tau protein. A particularly preferred disease for these embodiments is Alzheimer's disease. In some of these preferred embodiments, the disease is Alzheimer's and the analogous disease in a Drosophila is caused by alterations in expression or activity of presenilin or expression or activity of a component of the γ-secretase complex. Preferably, the disease is caused by expression of a mutant presenilin gene. See Example 3.

In still other preferred embodiments, the disease is caused by an expanded trinucleotide repeat, preferably an expanded glutamine repeat. Preferred examples include Huntington's disease and the analogous disease in a Drosophila is caused by an Htt exon1 protein with an expanded glutamine repeat. In additional preferred embodiments, the disease is Parkinson's disease and the analogous disease in a Drosophila is caused by an alteration in the activity or expression of α-synuclein. In preferred embodiments, the disease is caused by a mutant

These methods are expected to be useful for any metazoan subject to mental defects. Preferably, the metazoan is a mammal, most preferably a human.

These methods are also useful for screening any compound for the ability to reduce mental defects. In some preferred embodiments, the compound is an inhibitor of a glutamate receptor (GluR), preferably a metabotropic mGluR (mGluR), most preferably a group II or group III mGluR. The most preferred group II and group III mGluR inhibitors are selective for either GluR receptor, i.e., they do not significantly inhibit mGluRs of other groups at the concentration used. In other preferred embodiments, the compound is an inhibitor of an inositol trisphosphate receptor (InsP3R), a glycogen synthase kinase-3β (GSK-3β), or a phosphodiesterase-4 (PDE-4).

The compound can also be an organic compound less than 1000 Daltons or a nucleic acid, such as an antisense nucleic acid, a ribozyme, an aptamer, or an RNAi (e,g, an siRNA), which are well known in the art.

Several different mental defects in Drosophila can be measured to determine the effect of the compound on that defect. See Examples. Preferably, the measurement is of conditioned courtship behavior. Learning and memory in conditioned courtship behavior is preferably measured as a reduction in courtship index (CI) (see Examples). The conditioned courtship behavior can be, e.g., learning during training, immediate recall after training, short term memory at about 60 minutes after training, medium term memory, anesthesia resistant memory or long term memory or age dependent ability for learning during training, immediate recall after training, short term memory at about 60 minutes after training, medium term memory, anesthesia resistant memory, or long term memory.

In other embodiments, the invention is directed to methods of evaluating a compound for the ability to improve learning or memory in a mammal. The methods comprise determining whether the compound improves learning or memory in a Drosophila that is deficient in a dFMR1 or with altered function of at least one presenilin gene (see Examples). A preferred mammal in these embodiments is a human.

The invention is additionally directed to methods of improving learning or memory in a mammal. The methods comprise treating the mammal with a compound in an amount sufficient to improve learning or memory in the mammal. In these embodiments, the compound inhibits expression or activity of a group II or group III metabotropic glutamate receptor (mGluR), an inositol trisphosphate receptor (InsP3R), a glycogen synthase kinase-3β (GSK-3β), or a phosphodiesterase-4 (PDE-4) in the mammal. In preferred embodiments, the mammal has Fragile X syndrome, a tauopathy, a disease caused by a trinucleotide repeat such as Huntington's disease, Parkinson's disease, or a non-human disease analogous to Fragile X syndrome, a tauopathy, neurofibromatosis 1, Huntington's disease, or Parkinson's disease.

Preferred compounds in these embodiments are LiCl or an inhibitor of a group II or group III mGluR, where the inhibitor is at a concentration that it is specific for a group II or group III mGluR. Nonlimiting examples of compounds that inhibit a group II mGluR include 2-methyl-6-(phenylethynyl)pyridine (MPEP), 2-amino-4-phosphonobutanoic acid (AP-4), (R S)-α-methylserine-O-phosphate monophenyl ester, (RS)-1-amino-5-phosphonoindan-1-carboxylic acid [(RS)-APICA], (RS)-α-methyl-4-tetrazolylphenylglycine (MTPG), (2S)-α-ethylglutamic acid (EGLU), (2S)-2-amino-2-[(1S,2S)-2-carboxycycloprop-1-yl]-3-(xanth-9-yl) propionic acid (LY341495) and (RS)-alpha-methyl-4-phosphoonophenylglycine (MPPG). For (RS)-APICA, MTPG, EGLU, and LY341495, see www.tocris.com. Nonlimiting examples of compounds that inhibit a group III mGluR likewise include (RS)-α-methyl-4-tetrazolylphenylglycine (MTPG), (2S)-2-amino-2-[(1S,2S)-2-carboxycycloprop-1-yl]-3-(xanth-9-yl) propionic acid (LY341495), and MPPG in addition to MAP4. Nonlimiting examples of inhibitors of PDE-4 are 4-[3-(Cyclopentyl)-4-methoxyphenyl]-2-pyrrolidinone (rolipram), Ro 20-1724, Etazolate, RP 73401, and SB-207499. In preferred embodiments, the PDE-4 inhibitor is rolipram. Nonlimiting examples of inhibitors of GSK-3β are TDZD-8, and 1-azakenpaullone (Kunick et al., 2004). Other examples of compounds which can specifically inhibit group II or group III mGluRs, InsP3R, GSK-3β, or PDE-4 are nucleic acids such as antisense nucleic acids, a ribozymes, an aptamers, or RNAi specific for the group II or group III mGluR, the InsP3R, the GSK-3β, or the PDE-4. Such nucleic acids can be designed and synthesized without undue experimentation.

The above-described compounds can be formulated into pharmaceutical compositions without undue experimentation for administration to a mammal, including humans, as appropriate for the particular application. Additionally, proper dosages of the compounds can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compositions of the present invention can easily be administered parenterally such as for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

In these embodiments, the mammal is preferably a rodent (e.g., to determine the safety and efficacy of the compound in a mammal) or a human.

The present invention is also directed to methods of treating a mammal with Fragile X disease or a non-human disease analogous to Fragile X disease, or with altered function of at least one presenilin gene. The methods comprise treating the mammal with a compound in a pharmaceutically acceptable excipient, where the compound inhibits expression or activity of a group II or group III mGluR, InsP3R, a GSK-3β, or a PDE-4 in the mammal.

As in the embodiment described immediately above, preferred compounds in these embodiments are LiCl, MPEP, AP-4, (RS)-α-methylserine-O-phosphate monophenyl ester, RS)-APICA, MTPG, EGLU, LY341495, MPPG, MTPG, TDZD-8, and 1-azakenpaullone, MAP4, rolipram, Ro 20-1724, Etazolate, RP 73401, or SB-207499. In preferred embodiments, the PDE-4 inhibitor is rolipram. Other examples of compounds which can specifically inhibit group II or group III mGluRs, InsP3R or PDE-4 are nucleic acids such as antisense nucleic acids, a ribozymes, an aptamers, or RNAi specific for the group II or group III mGluR, the InsP3R, or the PDE-4. Such nucleic acids can be designed and synthesized without undue experimentation.

In these embodiments, the mammal is preferably a rodent (e.g., to determine the safety and efficacy of the compound in a mammal) or a human, e.g., with Fragile X syndrome.

An effective treatment in these embodiments preferably improves synaptic plasticity in the mammal, or improves the balance of long-term depression (LTD) to long-term potentiation (LTP) in the brain of the mammal (see Examples).

The inventors have also discovered that the same treatments that are effective in reducing mental defects, e.g., in learning and memory, in a Drosophila model of Fragile X syndrome are also effective in treatment of a Drosophila model of Alzheimer's disease. Particularly effective treatments here are compounds that inhibit group II or group III metabotropic glutamate receptors. See Example 3.

Thus, in additional embodiments, the invention is directed to methods of treating a mammal with Alzheimer's or a non-human disease analogous to Alzheimer's. The methods comprise treating the mammal with a compound that specifically inhibits expression or activity of a group II or group III mGluR, an InsP3R, a GSK-3β, or a PDE-4 in the mammal. In some embodiments, the mammal has mutations in the presenilin 1, presenilin 2 or APP genes, which can cause Alzheimer's.

Two Drosophila Alzheimer's models are particularly useful. One uses over expression of tau (either human wild type or Drosophila wild type with a myc tag) driven in the mushroom bodies. Flies overexpressing tau have normal memory at 5 days of age (young adults) but an age dependent impairment of short-term and long term memory by 26 days of age. Treatment with LY341495, MPEP, or lithium can rescue the age dependent short term memory deficit at 30 and 40 days in the wild type human and the Drosophila tau expressing flies. Additionally, LY341495 treatment performed from day 30 to day 39 can restore short term memory at 40 days of age.

The second particularly useful Drosophila Alzheimer's model utilizes a mutant presenilin gene. See Example 3.

In some preferred embodiments of these methods, the mammal is treated with LiCl.

Where the treatment is with an inhibitor of a group II or group III mGluR, the inhibitor is preferably selective for group II or group III mGluR (i.e., does not significantly inhibit group I mGluR), or is used at a concentration that is specific for a group II mGluR. As discussed above, examples of group II mGluR inhibitors are 2-methyl-6-(phenylethynyl)pyridine (MPEP), 2-amino-4-phosphonobutanoic acid (AP-4), (RS)-α-methylserine-O-phosphate monophenyl ester, (RS)-1-amino-5-phosphonoindan-1-carboxylic acid [(RS)-APICA], (RS)-α-methyl-4-tetrazolylphenylglycine (MTPG), (2S)-α-ethylglutamic acid (EGLU), and (2S)-2-amino-2-[(1S,2S)-2-carboxycycloprop-1-yl]-3-(xanth-9-yl) propionic acid (LY341495). Examples of group III mGluR include MAP4, (2S)-2-amino-2-[(1S,2S)-2-carboxycycloprop-1-yl]-3-(xanth-9-yl) propionic acid (LY341495), and (RS)-α-methyl-4-tetrazolylphenylglycine (MTPG); examples of inhibitors of GSK-3β are TDZD-8, and 1-azakenpaullone. Also as discussed above, nonlimiting examples of inhibitors of PDE-4 are 4-[3-(Cyclopentyl)-4-methoxyphenyl]-2-pyrrolidinone (rolipram), Ro 20-1724, Etazolate, RP 73401, and SB-207499.

The compound can also be a nucleic acid, such as an antisense nucleic acid, a ribozyme, an aptamer, or an RNAi that specifically inhibits expression or activity of the group II or group III mGluR the InsP3R, the GSK-3β, or the PDE-4, as previously discussed.

In these embodiments, the mammal is preferably a rodent or a human, most preferably a human. Effective treatments would be expected to improve synaptic plasticity in the mammal and/or improve the balance of long-term depression (LTD) to long-term potentiation (LTP) in the hippocampus of the mammal.

The present invention is additionally directed to kits for treating a mammal having Fragile X disease, neurofibromatosis 1, Alzheimer's disease, or a non-human analogy to Fragile X disease or Alzheimer's disease. The kits comprise (a) a compound in a pharmaceutically acceptable excipient, wherein the compound inhibits expression or activity of a group II or group III mGluR, an InsP3R, a GSK-3β, or a PDE-4 and (b) instructions directing the use of the compound for treating the mammal.

In preferred embodiments, the compound is a inhibitor of a group II or group III mGluR, for example 2-methyl-6-(phenylethynyl)pyridine (MPEP), and specific inhibitors such as 2-amino-4-phosphonobutanoic acid (AP-4), (RS)-α-methylserine-O-phosphate monophenyl ester, (RS)-1-amino-5-phosphonoindan-1-carboxylic acid [(RS)-APICA], (RS)-α-methyl-4-tetrazolylphenylglycine (MTPG), (2S)-α-ethylglutamic acid (EGLU), (2S)-2-amino-2-[(1S,2S)-2-carboxycycloprop-1-yl]-3-(xanth-9-yl) propionic acid (LY341495), or MAP4. In other preferred embodiments, the compound is LiCl or rolipram. Other nonlimiting examples of specific inhibitors of group II or group III mGluR, an InsP3R, a GSK-3β, or a PDE-4 are nucleic acids such as antisense nucleic acids, ribozymes, aptamers, or RNAi that specifically inhibits expression or activity of the group II or group III mGluR, the InsP3R, the GSK-3β, or the PDE-4. Such inhibitors can be made without undue experimentation.

In other embodiments, the invention is directed to the use of a compound for the manufacture of a medicament for the treatment of a mammal having Fragile X disease, neurofibromatosis 1, Alzheimer's disease or a non-human analogy to Fragile X disease or Alzheimer's disease. In these embodiments, the compound inhibits expression or activity of a group II or group III mGluR, an InsP3R, a GSK-3β, or a PDE-4. In preferred embodiments, the compound is an inhibitor of a group II or group III mGluR, such as 2-methyl-6-(phenylethynyl)pyridine (MPEP), or specific inhibitors such as 2-amino-4-phosphonobutanoic acid (AP-4), (RS)-α-methylserine-O-phosphate monophenyl ester, (RS)-1-amino-5-phosphonoindan-1-carboxylic acid [(RS)-APICA], (RS)-α-methyl-4-tetrazolylphenylglycine (MTPG), (2S)-α-ethylglutamic acid (EGLU), (2S)-2-amino-2-[(1S,2S)-2-carboxycycloprop-1-yl]-3-(xanth-9-yl) propionic acid (LY341495), or MAP4. Other preferred compounds are LiCl or rolipram, as discussed above. Other effective compounds in these embodiments are nucleic acids such as antisense nucleic acids, ribozymes, aptamers, or RNAi that specifically inhibits expression or activity of the group II or group III mGluR, the InsP3R, the GSK-3β, or the PDE-4.

Additionally, the present invention is directed to the use of a compound that inhibits expression or activity of a group II or group III mGluR, an InsP3R, a GSK-3β, or a PDE-4 in the treatment of a mammal having Fragile X disease, Alzheimer's disease, neurofibromatosis 1, or a non-human analogy to Fragile X disease, Alzheimer's disease or neurofibromatosis 1. Preferably, the compound is an inhibitor of a group II or group III mGluR, such as 2-methyl-6-(phenylethynyl)pyridine (MPEP), or the specific inhibitors 2-amino-4-phosphonobutanoic acid (AP-4), (RS)-α-methylserine-O-phosphate monophenyl ester, (RS)-1-amino-5-phosphonoindan-1-carboxylic acid [(RS)-APICA], (RS)-α-methyl-4-tetrazolylphenylglycine (MTPG), (2S)-α-ethylglutamic acid (EGLU), (2S)-2-amino-2-[(1S,2S)-2-carboxycycloprop-1-yl]-3-(xanth-9-yl) propionic acid (LY341495), or MAP4. In other preferred embodiments, the compound is LiCl or rolipram. Other useful compounds in these embodiments include nucleic acids such as antisense nucleic acids, ribozymes, aptamers, or RNAi that specifically inhibits expression or activity of the group II or group III mGluR, the InsP3R, the GSK-3β, or the PDE-4.

Preferred embodiments of the invention are described in the following Examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the Examples.

### EXAMPLE 1

### The Rescue of Synaptic Plasticity and Naive Courtship Behavior in the Drosophila Melanogaster Model of Fragile X Syndrome by Pharmacologic Treatment

### Example Summary

Fragile X mental retardation is caused by transcriptional silencing or the loss of the functional FMR1 gene product and is the leading heritable genetic cause of mental retardation. FMR1 is a known RNA binding protein, although the specific physiologic functions of FMR1 remain a mystery. Drosophila lacking functional dFMR1 protein exhibit reduced naive courtship level, arrhythmic circadian activity, erratic locomotor activity and altered transmission at the neuromuscular junction (Dockendorff et al, 2002; Zhang et al, 2001a). Here, we extend the model of Fragile X in Drosophila melanogaster to encompass synaptic plasticity, specifically addressing learning during training and memory at two distinct time points utilizing the conditioned courtship paradigm. We demonstrate that the Drosophila Fragile X protein is critical for synaptic plasticity of memory formation involved in experience dependent modification of courtship behavior. Moreover, we show that treatment by the compound 2-methyl-6-(phenylethynyl)pyridine (MPEP) at doses effective to antagonize the Drosophila group II metabotropic glutamate receptors (mGluRs) either in development, in adulthood or both can rescue naive courtship and synaptic plasticity in the form of memory in these mutant, FS, flies. Additionally, we demonstrate that treatment in adulthood with another group II mGluR antagonist, LY 341495, or with either 5 or 50 mM LiCl can restore naive courtship and synaptic plasticity in the form of memory in these mutant, FS, flies. These findings increase the utility of the Drosophila model, and raise the possibility that this compound or other compounds having similar effects the group II metabotropic glutamate receptors may rescue synaptic plasticity in Fragile X syndrome in humans.

### Introduction

In the conditioned courtship paradigm we demonstrated that the dFMR1 protein is not required for functional learning during training with a previously mated female. However, dFMR1 was required for behavioral plasticity immediately after training and 60 minutes after training with a previously mated female. Furthermore, in this paper we manipulated the group II/III mGluRs in the Drosophila model of Fragile X syndrome to rescue synaptic plasticity. This was accomplished by identifying and utilizing a conserved binding pocket for MPEP in the mGluRs which was used to antagonize the receptors via MPEP treatment and restore behavioral plasticity at the time points of immediate recall and short-term memory.

### Results

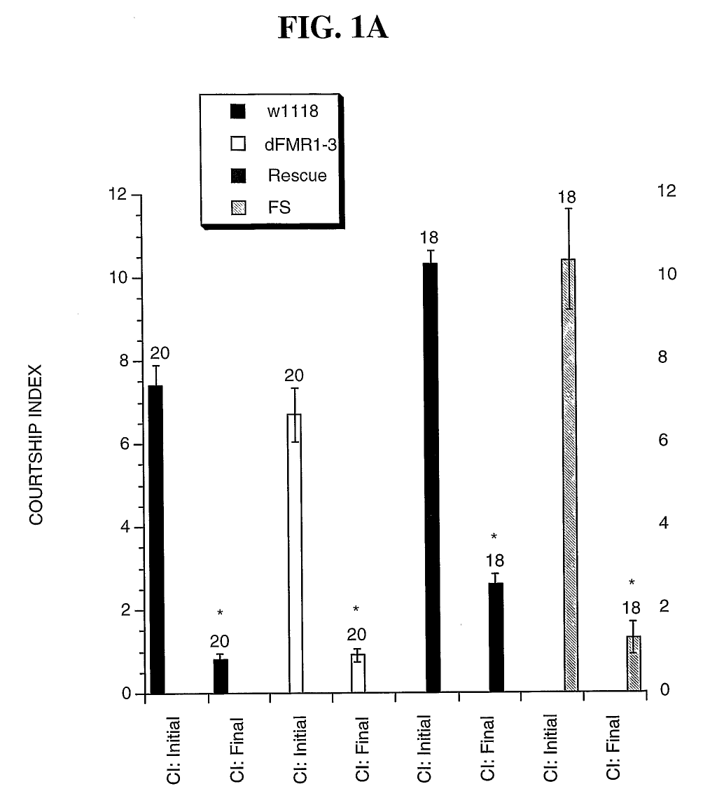

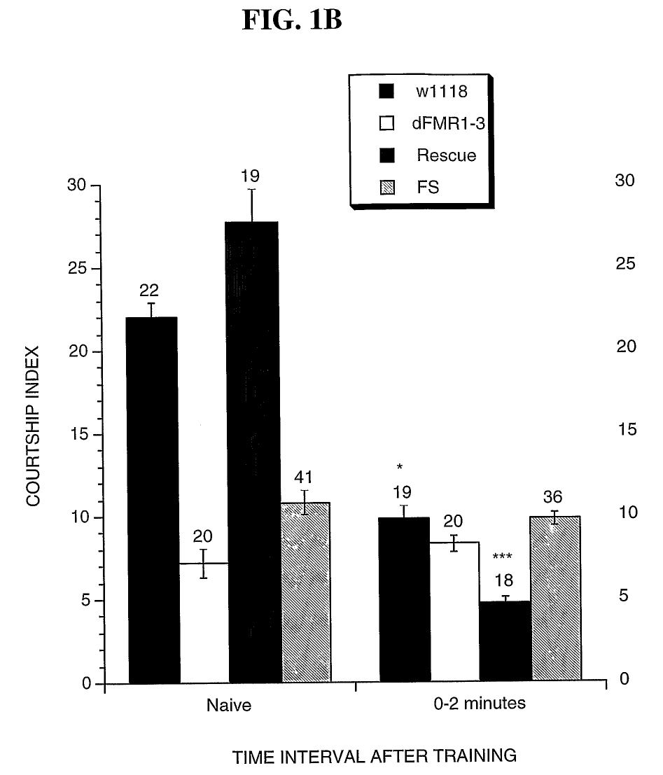

The role of dFMR1 in learning during training and immediate recall. The flies used were w1118 (the background stock from which the mutation was derived), dFMR1-3 (the mutation lacking dFMR1 expression), Rescue (dFMR1-3+wild type rescue fragment) and FS (frame shift, dFMR1-3+frame shifted rescue fragment) all from Dockendorff et al, 2002. To assess learning during training male flies are placed in a training chamber with a previously mated female for one hour. The amount of time the male spent courting in the initial ten-minute interval was compared to the amount of time the male spent courting the female target in the final ten-minute interval (FIG. 1). The initial and final courtship levels of w118 and dfmr1-3 are similar to each other and show significant depression from the initial to final intervals indicating that both groups demonstrated learning during training (p<0.005, FIG. 1A). The initial and final courtship levels of Rescue and FS flies are similar to each other and show significant depression from the initial to final intervals, likewise indicating that both groups demonstrated learning during training (p<0.005, FIG. 1A). It is important to note that the level of courtship behavior towards the previously mated female is similar between the two mutant groups and the w1118 and Rescue groups. This indicates that although naive courtship level has previously been shown to be depressed in the two mutant groups (Dockendorff et al, 2002), there is enough courtship activity present to adequately train each of the two mutant groups. This is important to note, because without actively courting, the male fly cannot be trained (Tompkins et al. 1982; 1983). Since naive courtship has been shown to be depressed in the two mutant groups, and olfactory acuity, visual acuity and locomotor activity can affect courtship, all of these abilities were assayed and found to be similar to w1118 and Rescue lines (Dockendorff et al, 2002).

To assess immediate recall (0 minute memory), after the one hour training session with a previously mated female, the male was placed with a virgin target female for a ten-minute interval. This CI was then compared to the courtship level of naive males that had been placed in the training chamber for one hour with no female, before introducing them to a virgin target female for a ten-minute interval. In FIG. 1B the w1118 and Rescue lines show depression of courtship activity after training compared to naive trained males (p<0.005). However, dFMR1-3 and FS mutant lines court just as vigorously after training with a previously mated female as naive trained males, therefore these flies display no experience dependent behavioral plasticity at the time point of immediate recall. This is in spite of the fact that they court to similar levels as w1118 and Rescue lines during the training period. This implicates a deficit in synaptic plasticity for the two mutant lines at immediate recall (0 minute memory). This robust deficit in behavioral plasticity is a critical extension of the previous models of Fragile X syndrome in model organisms, since this has been completely missing in Drosophila models and is subtle in murine models, and particularly since this may be one of the most devastating aspects of the human disease.

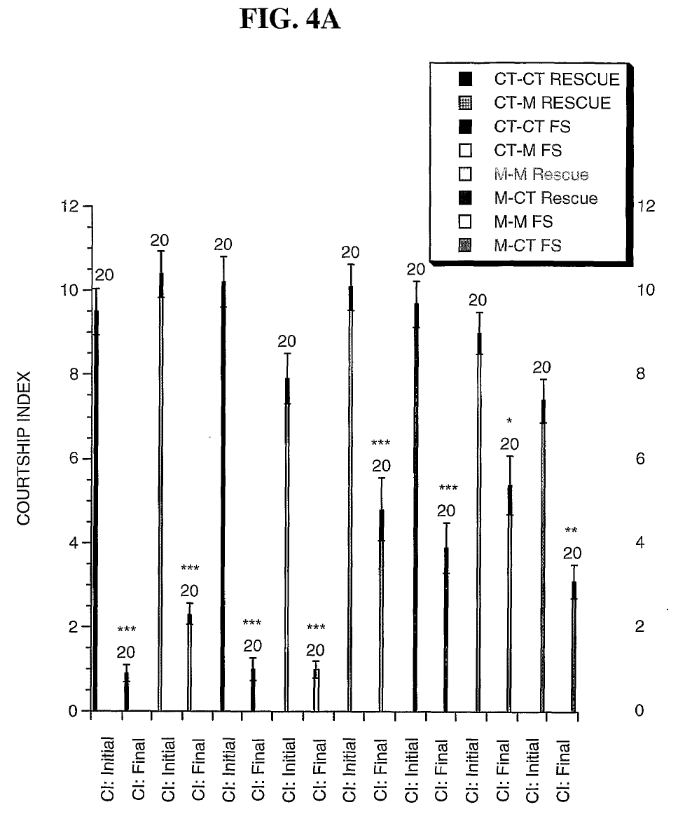

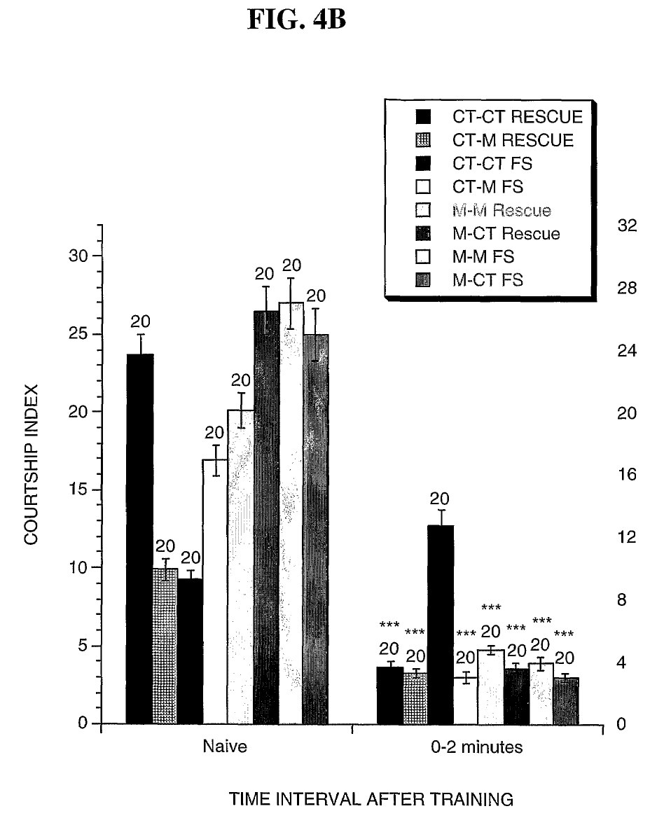

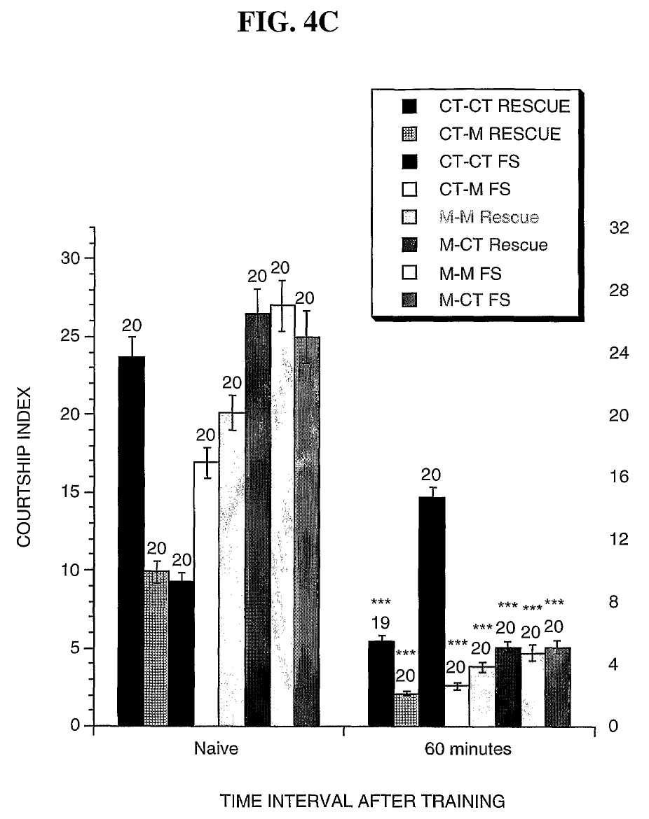

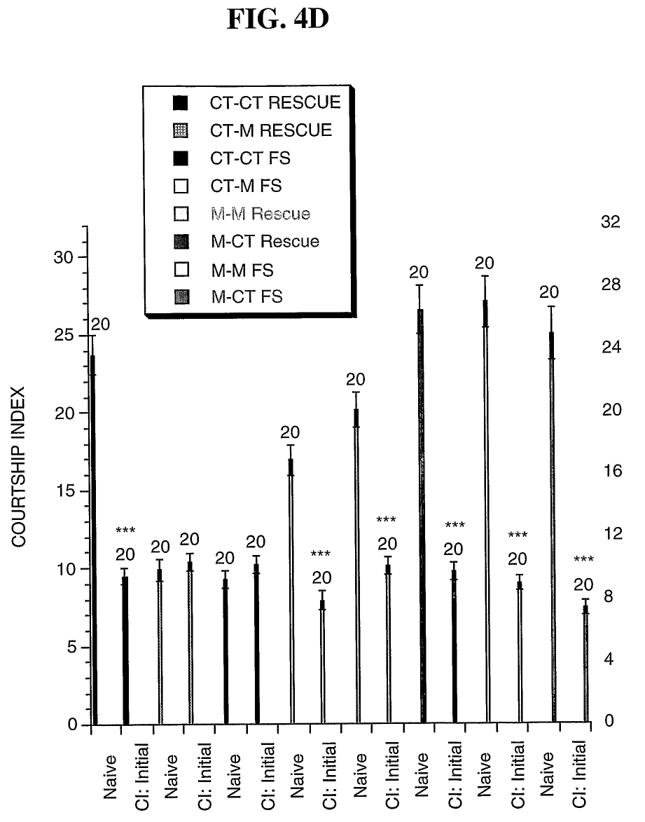

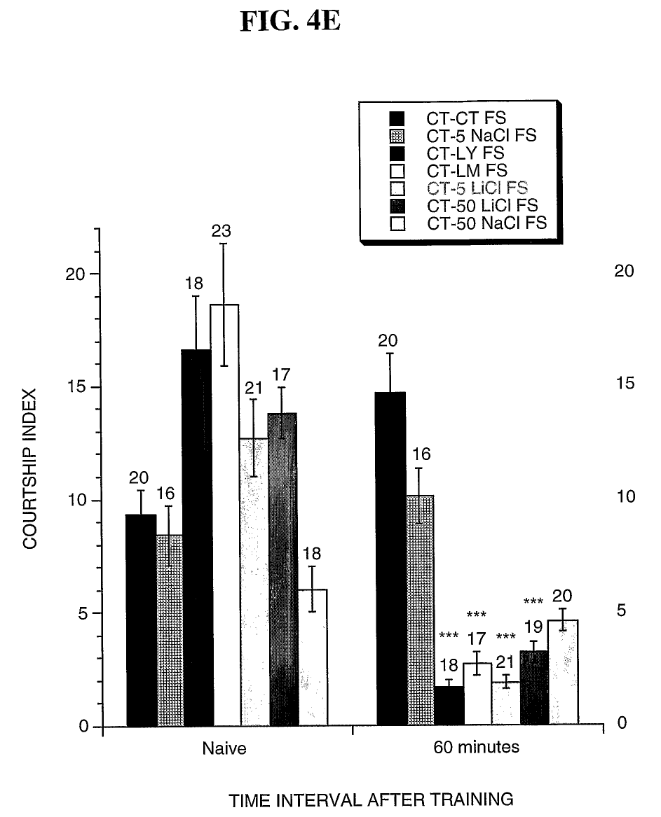

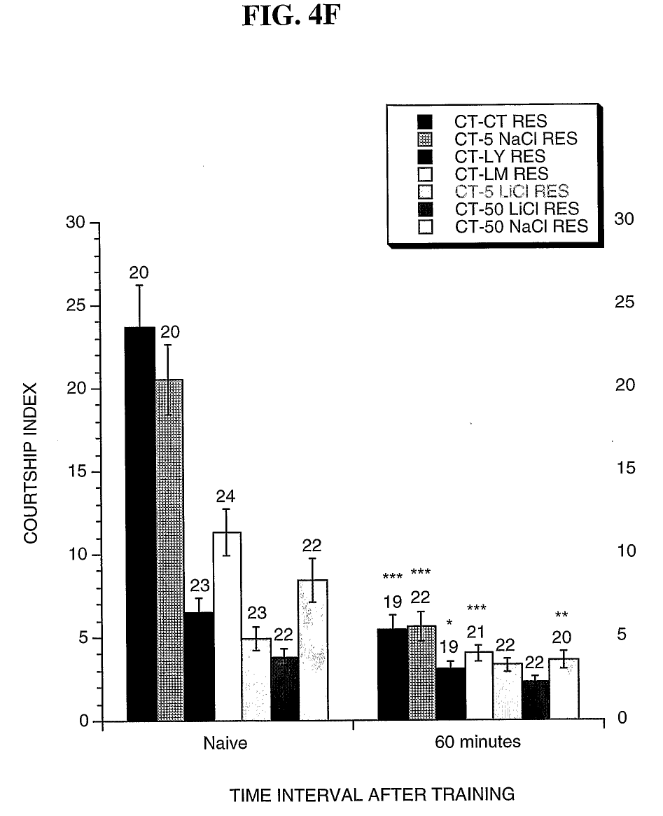

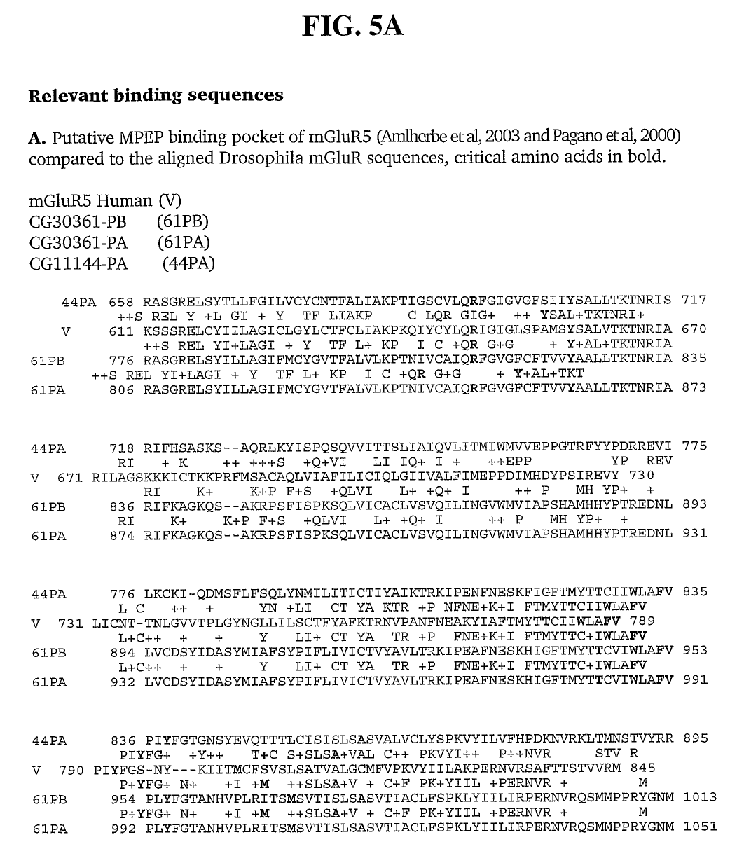

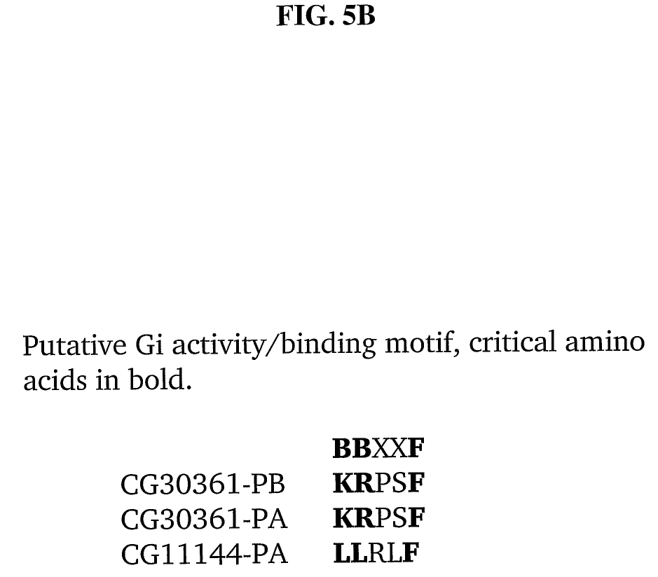

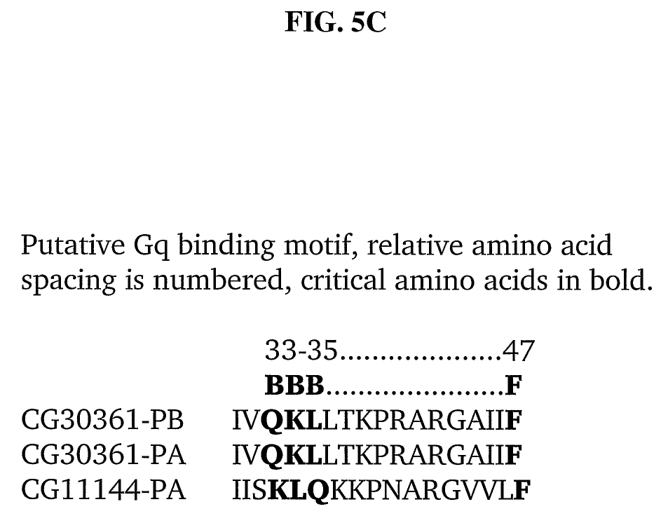

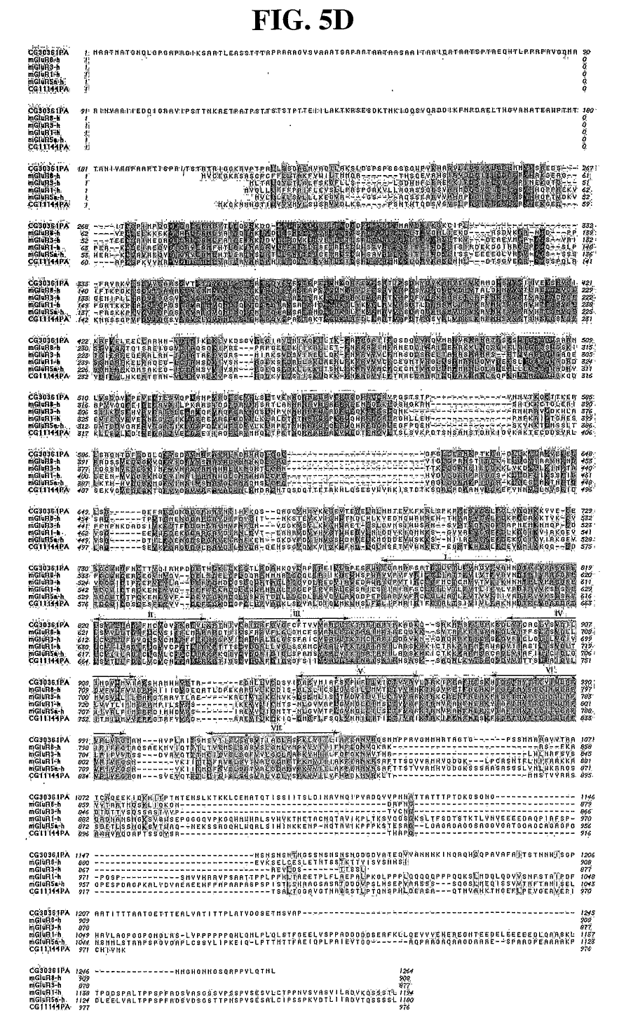

Since we use female targets that are not etherized (therefore they are freely moving), and the w118 and dfmr1-3 flies may have slightly impaired visual acuity due to lack of eye pigmentation, and since visual inputs affect courtship behavior, all subsequent experiments were done utilizing only the genotypes Rescue: dfmr1-3+wild type rescue fragment, and FS: dfmr1-3+frame shifted rescue fragment. Both of these genotypes have more pigmentation and similar levels of eye pigmentation to each other. For FIGS. 4 and 5, a high concentration of MPEP (1,000 μg/ml) was used. For the remainder of the experiments the food was either control (CT) or exactly the same control food containing the appropriate concentration of MPEP (M). The position of the CT and the M are indicative of the point at which the group was on the particular food. The first letter indicates the food type that the larvae grew up on, and the second letter denotes the food type that the adult flies were placed on within four hours of eclosion, and for the four following days, until the day before testing when they were placed on fresh food.

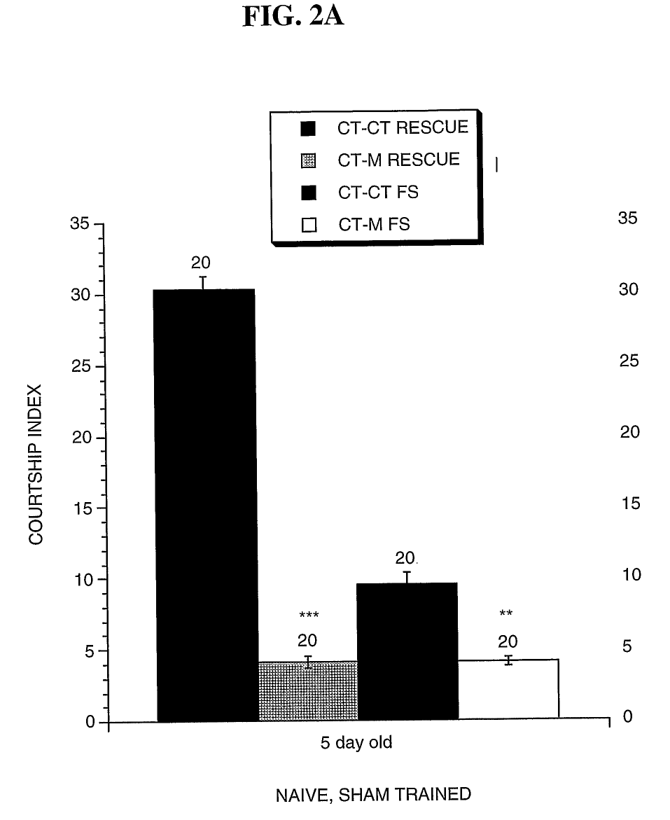

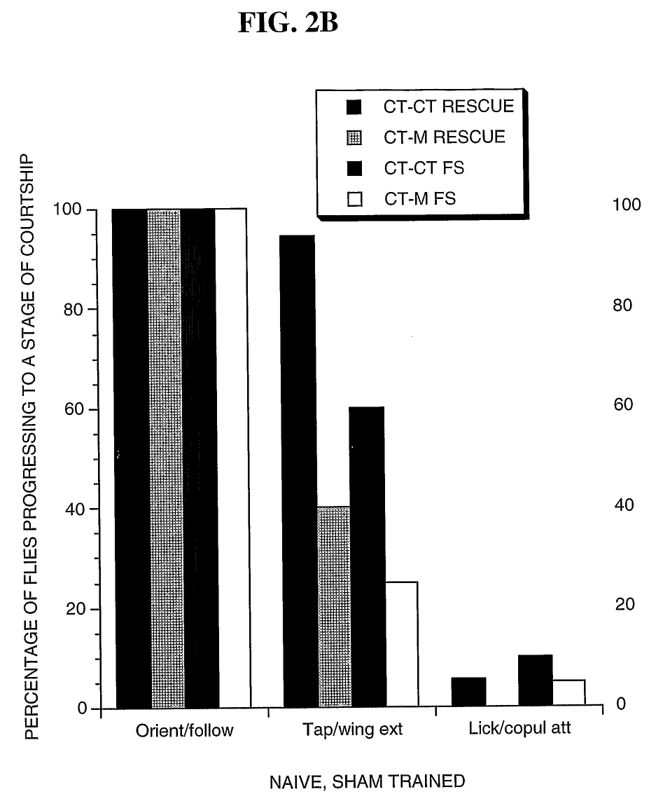

In order to determine if MPEP can get into the Drosophila central nervous system (CNS) through feeding behavior, we mixed MPEP into the food at a concentration of 1,000 μg/ml (433.0 μM). Males were raised on CT food and then placed on either CT or M food. Naive males were placed in the training chamber for one hour with no female, and then placed with a virgin target female for a ten-minute interval. The naive courtship behavior was reduced by high MPEP concentration in Rescue (p<0.0001) and FS (p<0.0005) lines, and it was depressed to similar levels by high MPEP treatment in the Rescue and FS groups (FIG. 2A). This demonstrated that the drug was getting into the Drosophila CNS and presumably hitting a receptor, either one of the two mGluRs that are closely related to human group II/III mGluRs. FIG. 2B dissects the steps involved in naive courtship behavior. The CT-CT FS group along with the two high MPEP treated groups failed to progress to later steps in courtship behavior relative to the CT-CT Rescue group, again demonstrating that this high dose of MPEP inhibits courtship activity.

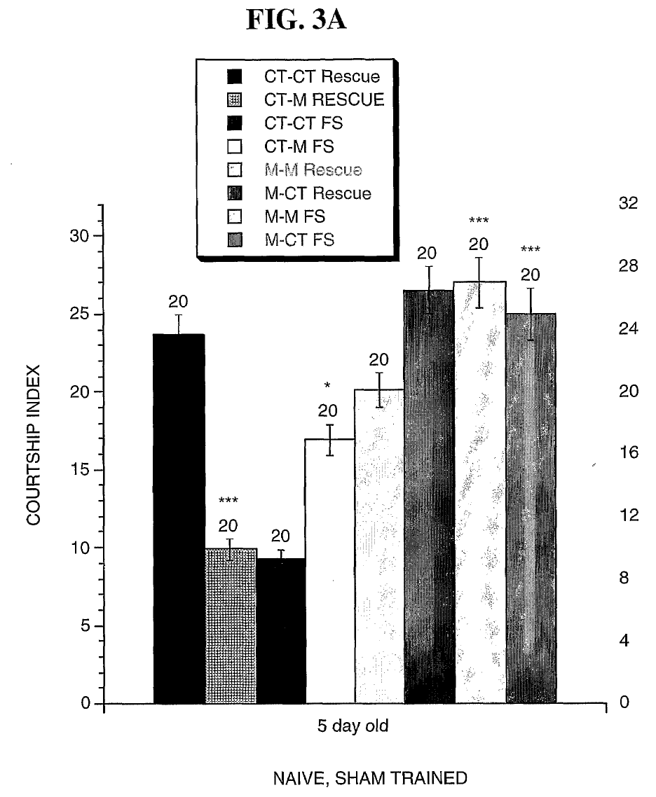

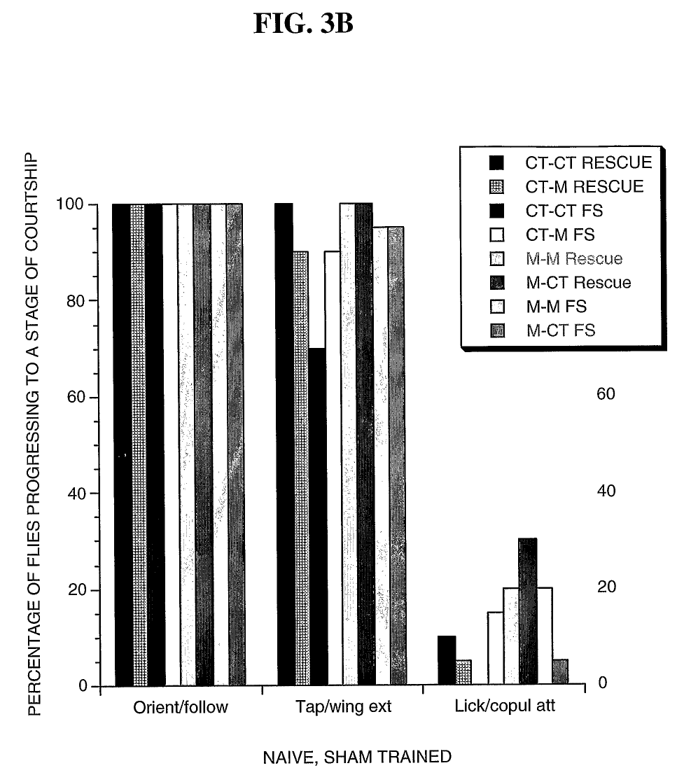

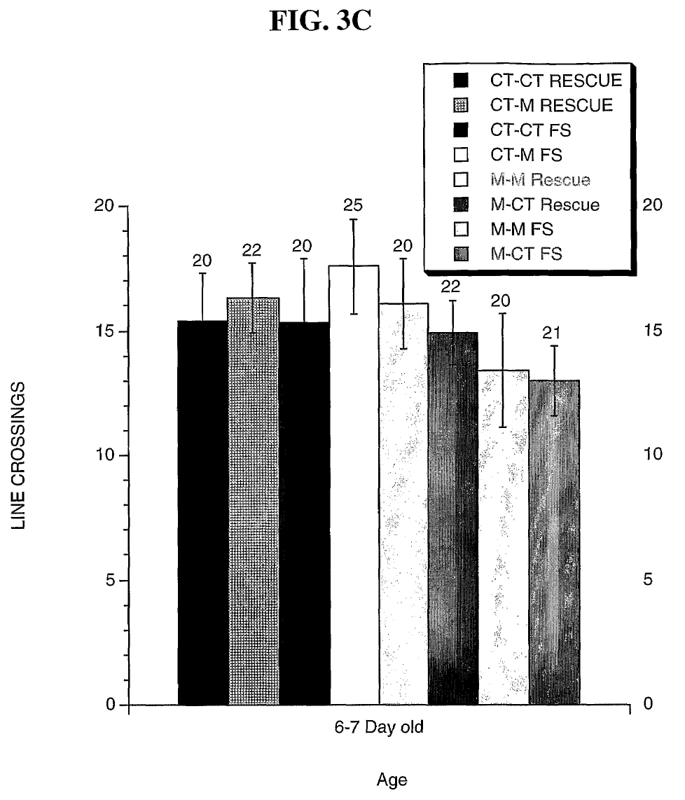

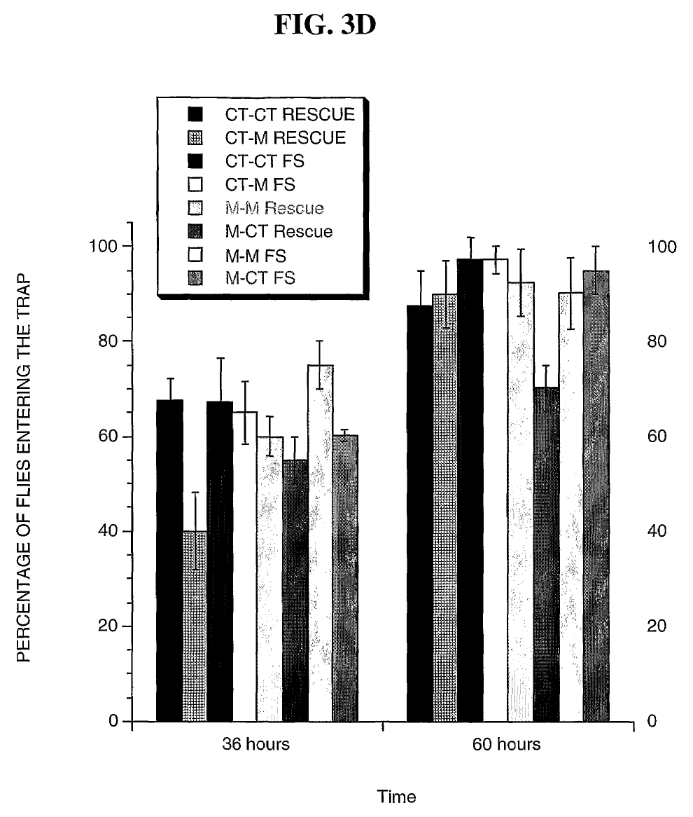

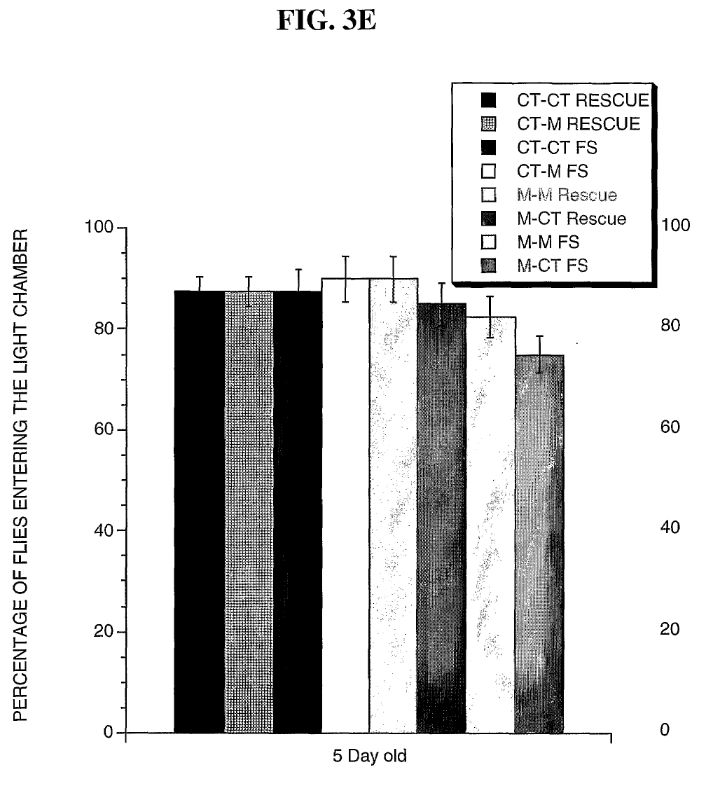

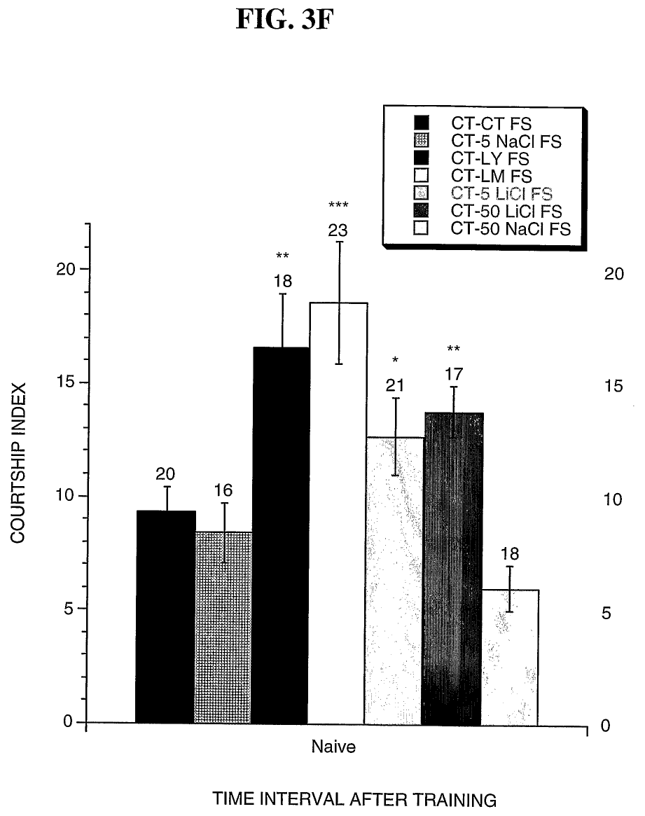

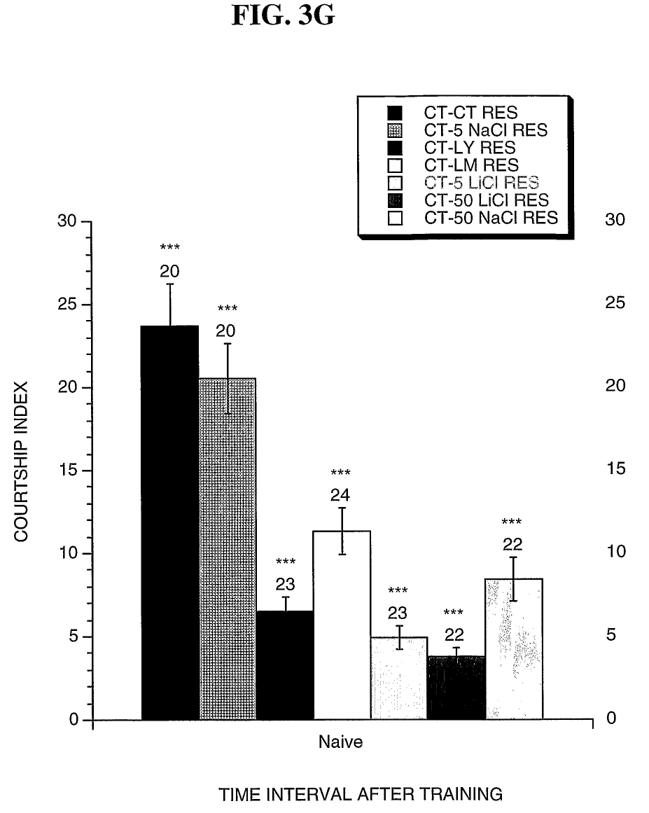

Lowering the dose of MPEP and examining naive courtship. We next decided to try a lower dose of MPEP to 200 μg/ml (86.6 μM) mixed into the food of the Drosophila. In these experiments flies were placed on either CT or M food in the larval stage and then placed on either CT of M food within four hours of eclosion. When Rescue flies were raised on CT food and then placed on M food as adults, courtship activity was depressed relative to CT-CT Rescue flies (p<0.0001), therefore giving this drug to healthy flies is not without some side effects (FIG. 3A). FS flies raised on C food and then placed on M food as adults show a significant increase in courtship activity relative to CT-CT FS flies (p<0.005, FIG. 3A). Therefore giving this drug in adulthood only is effective in enhancing naive courtship behavior. Rescue and FS flies on M food in development and then placed on either M or CT food as adults courted as vigorously as CT-CT Rescues flies. This shows that development in the presence of M food with or without M food as adults is able to restore naive courtship behavior in FS flies. It also shows that during development the effect of the drug on disturbing the courtship activity on healthy Rescue flies can be compensated for, so that if the Rescue lines have it in development naive courtship remains intact regardless of whether they receive M food as adults. Therefore, M-M FS and M-CT FS groups show courtship levels similar to CT-CT Rescue flies. The MPEP in development can rescue the naive courtship phenotype regardless of whether or not the flies receive it as adults. Further analysis of the quality of courtship that was performed by naive males was assessed by binning the number of males to advance to a particular phase of courtship for each genotype and pharmacologic treatment (FIG. 3B). Even though the CT-M rescue flies showed a low amount of time involved in courtship as naive flies, they still progressed to later phases of courtship to similar levels as all other groups excepting the CT-CT FS group. A higher percentage of the CT-CT FS group failed to advance to later stages of courtship compared to all other groups. This demonstrates the in development or adulthood, MPEP treatment can affect the quality of naive courtship behavior in FS flies. Locomotion was assayed and found to be similar in all groups (FIG. 3C). Visual acuity was assayed and found to be similar in all groups (FIG. 3D). Olfaction was also assayed and found to be similar in all groups (FIG. 3E).

Panel F shows the effect of LiCl (5 mM and 50 mM), MPEP (20 μg/ml) and the group II selective mGluR antagonist LY341495 (400 nM) on FS naive courtship, all of which significantly increased naive courtship while NaCl at 5 or 50 mM did not restore naive courtship. Panel G shows the effect of LiCl (5 mM and 50 mM), MPEP (20 μg/ml), LY341495 (400 nM) and NaCl (50 mM) on rescue naive courtship, all of which suppressed naive courtship, although 5 mM NaCl did not suppress naive courtship.

Learning during training. In order to assess learning during training, the male flies were placed in a training chamber with a previously mated female for one hour. The amount of time the male spent courting in the first ten-minute interval was compared to the amount of time the male spent courting the female target in the last ten-minute interval (FIG. 4A). The initial and final courtship levels of all groups show significant depression from the initial to final intervals indicating that all groups demonstrated learning during training (CT-M Rescue, p<0.0001, CT-CT Rescue, p<0.0001, CT-M FS, p<0.0001, CT-CT FS, p<0.0001, M-M Rescue, p<0.0001, M-CT Rescue, p<0.0001, M-M FS, p<0.005, M-CT FS, p<0.0005). Additionally, since naive courtship is depressed in the two groups CT-M Rescue and CT-CT FS, it is important to note that the level of courtship behavior towards the previously mated female is similar to CT-CT Rescue and CT-M FS groups, indicating that enough courtship activity is present to adequately train each of these two groups. This demonstrated that treatment by MPEP in development, adulthood or both does not adversely affect learning during training, which is normally intact even in FS flies raised on solely CT food (see also FIG. 1A).

Immediate recall. After the one training session with a previously mated female, the male was placed with a virgin target female for a ten-minute interval. The male behavior was then compared to the courtship of naive males placed in the training chamber for one hour with no female, and then placed with a virgin target female for a ten-minute interval (see FIG. 4B). The CT-M Rescue line shows depressed courtship activity immediately after training (p<0.0001), indicating that M food as adults is not impairing behavioral plasticity in these flies, although it did depress naive courtship. This is also critical because since this group had very similar naive courtship as the CT-CT FS flies, it shows that the behavioral plasticity of the CT-CT FS group is not missed due to an artifact of low naive courtship. That along with the fact that the CT-CT FS group courts to similar levels in the first and last ten minute intervals of training with previously mated females as CT-M Rescue, CT-CT Rescue and CT-M FS lines demonstrates that the CT-CT FS flies were receiving adequate training and had a high enough naive courtship level to see a reduction in courtship activity after training if it were to occur. It did not occur, since CT-CT FS flies courted just as vigorously immediately after training as naive CT-CT FS flies (FIG. 4B). This again demonstrates that no experience-dependent behavioral plasticity occurs in CT-CT FS flies, as was also seen in FIG. 2. All Rescue groups demonstrate depression of courtship activity immediately after training relative to group matched naive flies to the level of p<0.0001. This indicates that M food in development or adulthood or both does not adversely affect immediate recall in rescue groups. The remaining FS groups that were treated with MPEP all display experience-dependent reduction of courtship activity immediately after training when compared to group matched naives, CT-M FS, p<0.0001, M-M FS, p<0.0001, and M-CT FS, p<0.0001 (FIG. 4B). Therefore, treatment with MPEP in development alone, in adulthood alone, and treatment in development and adulthood together are all sufficient to rescue synaptic plasticity in FS flies.

Short-term memory. The model was further extended to encompass short-term memory at 60 minutes after training. After the one hour training session with a previously mated female, the female is removed and the male is placed in a holding chamber for 60 minutes, then subsequently placed in a testing chamber with a virgin female target to assess short-term memory (FIG. 4C). The CT-M Rescue line shows depressed courtship activity at 60 minutes after training (p<0.0001), indicating that M food as adults is not impairing behavioral plasticity at 60 minutes post training in these flies, although it did depress naive courtship. CT-CT FS flies court just as vigorously at 60 minutes after training as naive CT-CT FS flies. This demonstrates the absence of short-term memory in CT-CT FS flies. Hence now the model has been further extended to encompass short-term memory. The Rescue groups treated with MPEP in development alone, in adulthood alone or in both development and adulthood demonstrate depression of courtship activity at 60 minutes after training relative to group matched naive flies (p<0.0001; FIG. 4C). This indicates that M food in development, adulthood or both development and adulthood does not adversely affect short-term memory in Rescue groups. The FS groups that were treated with MPEP in development alone, adulthood alone or in both development and adulthood display experience dependent reduction of courtship activity at 60 minutes after training when compared to group matched naives M-M FS, p<0.0001, CT-M FS, p<0.0001 and M-CT FS, p<0.0001. Therefore short-term memory is also rescued in FS flies by MPEP treatment. Additionally, flies were tested for ability to discriminate between virgin and previously mated females (FIG. 4D). Normally a wild-type naive male displays less courtship when paired with a previously mated female target compared to when paired with a virgin female target. This is the case for all groups except CT-M Rescue and CT-CT FS, which spend similar amounts of time courting previously mated female targets and virgin female targets (FIG. 4D). This phenomenon, however, does not seem to be critical for behavioral plasticity at 0 minutes or 60 minutes after training since the CT-M rescue group is lacking this phenomenon but does display memory at these two time points.

In tests of FS short term memory, LY341495, 5 mM and 50 μM LiCl, and MPEP restored short-term memory, whereas NaCl had no effect (FIG. 4E). MPEP, NaCl and LY341495 did not affect short term memory in Rescue flies, however LiCl treatment appeared to disrupt short term memory in Rescue flies (FIG. 4F).

### Discussion

Model of the dysfunctional anatomical areas in mutant flies. It was previously found that dFMR1 expression was needed for normal levels of naive courtship activity in response to a virgin female target (Dockendorff et al, 2002). We have discovered here that dFMR1 expression is not required for a normal response to a previously mated female target. Additionally, the protein is not required for learning during training to occur when paired with a mated female over the course of one hour. Therefore, working memory and behavioral plasticity was left intact. However, when paired with a virgin female 0 minutes after training, there is no evidence of memory of the training experience at the immediate recall time point. Furthermore, this is apparently not due to a deficit in any sensory modality or motor ability. In our mutant flies there is a global lack of dFMR1 protein, just as there is in the human manifestation of the disease. Therefore, to try to determine the critical brain regions that may be adversely affected by this lack of dFMR1 with regard to our testing paradigm, we tried to place these results in the context of previous findings on the anatomical substrates for learning during training and memory.

Dujarin (1850) first proposed the involvement of the mushroom bodies (MBs) in memory citing the similarities of the structural features to the structural features of the human hippocampus. Erber et al. (1980) used cooling experiments which lesion mainly the mushroom bodies (but other structures as well) to link the MBs to olfactory memory in Apis mellifera (honeybees). Additionally, Heisenberg (1980) utilized the mushroom body deranged mutation to demonstrate that there was an impairment of 0 minute memory of conditioned courtship. However, again, the mutation affected many areas of the brain. In Drosophila, the MBs arise from bilateral clusters of about 2,500 Kenyon cells located in the dorsal and posterior cortex (Davis, 1993; Strausfeld et al, 1995, Yang et al, 1995). Information from various sensory systems including olfactory, gustatory, visual and thoracic sensory systems feed into the MBs, making them an ideal candidate to form associations from various environmental stimuli (Powers, 1943; Strausfeld, 1976; Schildenberger, 1984; Strausfeld et al, 1995; Heisenberg et al, 1994; Barth and Heisenberg, 1997; McBride et al, 1999). The antennal lobes (AL) were shown to be involved in memory out to 30 minutes post training and the optic lobe (OL) was also speculated to have a similar ability for plasticity in honeybees and fruit flies (Faber et al, 1999; McBride et al, 1999). Short-term memory at the 60 minute time point was isolated to the MBs in an ablation experiment utilizing the conditioned courtship paradigm (McBride et al, 1999). This result was later confirmed in the olfactory association paradigm (Zars et al, 2000; Dubnau et al, 2001; McGuire et al, 2001). The MBs were also shown to be required for long-term memory formation in two novel conditioned courtship conditioning training paradigms (McBride et al, 1999), which was again confirmed in the olfactory association paradigm (Pascual and Preat, 2001). The dFMR1 mutant flies have a deficit in memory that is apparent at the 0 minute time point (immediate recall). In accordance with our previous model this would be consistent with dysfunction in the AL, OL or perhaps the calyces of the MBs. If the mushroom body beta and gamma lobes are seen as the association center analogous to the hippocampus in mammals, then the antennal lobe would be acting like the olfactory bulb and a portion of the antennal lobe and the calyces/alpha lobe of the MBs would be analogous to the prefrontal cortex. Additionally, our previous finding that attention may be altered in dFMR1 knockout flies evidenced by shortened courtship bouts, fits well with this model because the prefrontal cortex is known to modulate attention (Goldman-Rakic, 1995).

A strategy to rescue function. The activation of metabotropic glutamate receptors (mGluRs, the subtype was not characterized) by the addition of exogenous glutamate has been shown to induce an intracellular calcium rise and PKC activation dependent form of long-term depression (LTD) leading to the synthesis of FMR1 protein in mice (Weiler and Greenough, 1999). The FMR1 knockout mice also have enhanced LTD in the hippocampus as a result of increased group I subtype 5 in mGluR activity, although in most cases long-term potentiation (LTP) has been found to be unaffected in the hippocampus of knockout mice (Huber et al, 2002, Paradee et al., 1999, Li et al., 2002 and Godfraind et al., 1996). This finding of intact LTP is in spite of the fact that humans suffering from Fragile X syndrome have lowered cyclic AMP (cAMP) production (Bakker and Oostra, 2003). However, in the cortex Li et al (2002) found a significant reduction in LTP in the prefrontal cortex of knockout mice along with reduced expression of the ionotropic glutamate receptor AMPA. One of the proteins suppressed by FMR1 is the inositol trisphosphate receptor (InsP3R) (Darnell et al., 2001). The InsP3R is involved in the modulation of cytoplasmic free calcium concentration which plays a role in intracellular signaling that regulates a diverse set of physiologic processes in cells (Mak et al., 1998). The InsP3R is linked to TRP channels and group I mGluRs via an interaction with the adapter protein homer and has been shown to be involved in the establishment of LTD (Brakeman et al, 1997, Yuan et al, 2003, Khodakhah and Armstrong, 1997, Nakamura et al, 1999).

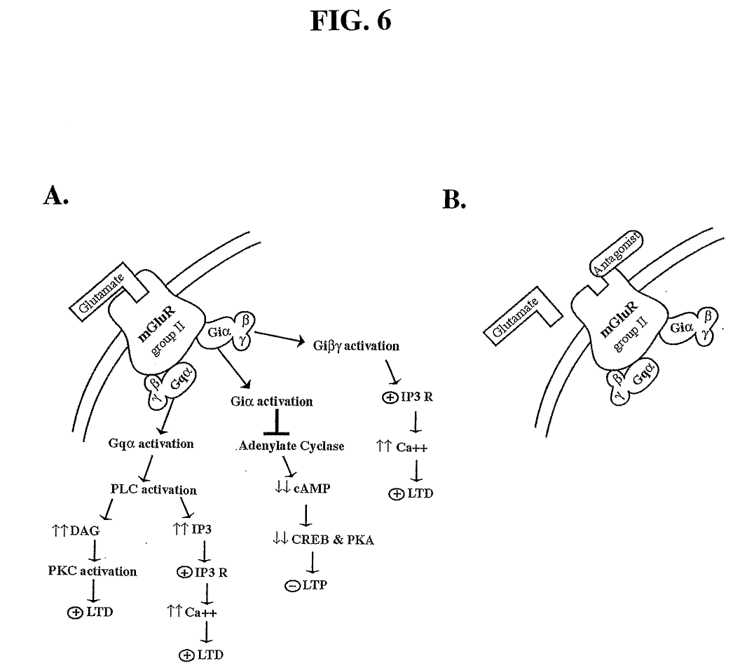

In order to reestablish the proper balance of LTD and LTP in our dFMR1 knockout flies to restore synaptic plasticity as evidenced by immediate recall and short-term memory at 60 minutes, we first attempted to lower InsP3 concentration and therefore InsP3R activity. We treated Rescue and FS flies with 50 mM LiCl since that dose was shown to lower InsP3 concentrations in cells and is an effective dose at which to inhibit InsP3R mediated neurite extension (Berridge et al, 1989, Berridge, 1993 and Takei et al, 1998). Additionally, LiCl treatment has been shown to enhance LTP in mice (Son et al, 2003). This was effective in increasing the levels of naive courtship in mutant flies, but not up to the level of controls (data not shown). LiCl treatment hits many targets within cells and we wanted to use something with greater specificity. In the Drosophila genome, only two mGluRs are present, one containing two predicted spliceoforms. One of these has previously been shown to be group II, and the other also bears closest homology to group II and then group III mGluRs (Parmentier et al, 1996 and Raymond et al, 1999). The sequences CG30361-PB and CG30361-PA, which are spliceoforms of the same gene, are most closely related to, in order, human mGluR isoforms 8, 7, 4, 2, 3, and 6 by amino acid identities ranging from 42-40% and amino acid conservation ranging from 62-58% (FIG. 5A). CG 11144-PA is most closely related to, in order, human mGluR isoforms 3, 2, 7, 8, 4, and 6 by amino acid identities ranging from 47-43% and amino acid conservation ranging from 63-59% (FIG. 5A). The previously characterized receptor termed DmGluR-A is expressed in the optic lobes, antennal lobes, the calyces, the central complex and the median bundle (Ramaekers et al, 2001). There is a stretch of sequence that shows conservation for Gi activation and binding motif in each sequence (Wade et al, 1999, FIG. 6B). Three lines of reasoning encouraged us to pursue this angle to modulate the balance of LTD/LTP. Normally group II mGluRs are coupled to Gi which negatively modulates adenylate cyclase (AC) activity in Drosophila and mammals, and indeed dysfunctional adenylate cyclase activity has been shown to impair learning and memory in the Drosophila mutant rutabaga (Levin et al, 1992, and Wang and Storm, 2003). By antagonizing group II mGluRs in Drosophila the inhibitory effect on adenylate cyclase activity would be decreased, thereby increasing AC activity and increasing cAMP levels and CREB activity presumably increasing the sensitivity to LTP. CREB activity in Drosophila and mammals is involved in memory formation and altering the isoform or activity of CREB can enhance memory in Drosophila and additionally enhance LTP in Aplysia and mice (Yin et al, 1994, Yin et al, 1995, Vitolo et al, 2002, Bozon et al, 2003 and Chen et al, 2003). Second, the beta and gamma subunits of Gi, which associate with group II mGluRs, bind the InsP3R and activate it without the need of InsP3 generation (Zeng et al, 2003). Therefore, inhibiting the activity of the beta and gamma subunits of Gi may disrupt a critical signaling process that is necessary to modulate InsP3R activity in a manner by which to establish LTD. Third, the prefrontal cortex is implicated in attention and memory guided behavior in primates (Goldman-Rakil, 1995). This is the area that we believe is analogous to the AL and calyx in our model, and represents our deficit in immediate recall. New evidence is emerging that in the prefrontal cortex there are postsynaptic group II mGluRs, in contrast to earlier studies (Petralia et al, 1996, Otani, 2002). These group II mGluRs can induce LTD in a manner dependent on PLC and IP3R activity (Haung et al, 1999a, Haung et al, 1999b, Otani et al, 1999 and Otani et al, 2002). There is also an interplay between the activity of group II mGluRs affecting the activity of NMDA receptors and vice versa, and that NMDA activity can affect the ability of group II mGluRs to induce LTD (Cho and Bashir, 2002). This may indicate a Gq binding site in addition to the Gi binding site in these receptors. There are other examples of metabotropic receptors binding multiple isoforms of G proteins (Wade et al, 1999). Additionally, there is a conserved putative binding site for Gq in Drosophila group II mGluRs (Pommier et al, 2003, FIG. 5C). Therefore, we felt that altering the activity of the group II mGluRs in Drosophila was an attractive candidate by which to modulate synaptic plasticity.

Since not much has been studied about these mGluRs in Drosophila, we next chose to look at mGluR antagonists that have been well studied and whose binding pockets have been characterized, then to determine whether the binding pocket is conserved when we align the sequences. MPEP is a mammalian group I subtype 5 mGluR antagonist, that has a well characterized binding pocket (Pagano et al, 2000 and Malherbe et al, 2003). This binding pocket is conserved in the Drosophila mGluR group II receptors in the appropriate putative secondary structure (see FIG. 5A). To ensure that these are the only putative targets of the drug, we blasted chunks of the binding pocket to see if any other proteins in Drosophila showed homology in this region. Only GABA receptors appeared to have homology in this region, although the GABA receptors did not show conservation of the residues that have been shown to be critical for MPEP binding (data not shown).

We initially determined that a high dose of 433 μM of MPEP could affect the courtship behavior of control and mutant flies, resulting in a significant decrease in courtship behavior. We then lowered the dose of MPEP in the food to 86.6 μM. Rescue flies that received MPEP in adulthood only, exhibited decreased naive courtship behavior. Rescue flies that received MPEP in development alone or development then adulthood did not show diminished naive courtship activity. In rescue flies working memory and memory at 0 minutes and 60 minutes after training remained intact irrespective of treatment by MPEP. Treatment in development, adulthood or both with 86.6 μM MPEP in the food significantly increased naive courtship levels of FS flies lacking any dFMR1 protein expression. Treatment in development, adulthood or both can also restore the quality of naive courtship in FS lines. After treatment by MPEP in development, adulthood or both, synaptic plasticity as evidenced by behavioral plasticity in the form of a suppression of courtship activity at immediate recall is restored in FS flies to a level similar to that which is displayed by Rescue flies. Additionally, after treatment in development, adulthood or both, short-term memory at 60 minutes after training is exhibited by FS flies to a level similar to that which is displayed by Rescue flies.

Examination of the MBs and antennal lobes had previously revealed no identifiable differences in morphology between mutant and control flies (Dockendorff et al, 2002). Here we further investigated whether there was any apparent difference in the dendritic morphology of mutant flies. We then examined the effects of MPEP treatment in adulthood in mutant flies. It is noteworthy that treatment only in development as larvae, or only in adulthood can fully restore memory in this paradigm, which may have implications on how we view this disease and the treatment of this disease in humans. A diagram of a proposed model of how this may be occurring upon treatment with MPEP is shown in FIG. 6.

We have extended the Drosophila model of Fragile X syndrome to now include a phenotype that is at the heart of the disease in humans, and in an ethologically relevant learning and memory paradigm in Drosophila. This phenotype may have certain advantages in both cost effectiveness in conducting screens for modulation of phenotype in Drosophila versus mice, and in robustness of the phenotype in Drosophila versus mice. In addition, we have identified novel targets for therapeutic intervention that are applicable in this model and may be applicable in other neurological disorders primarily involving learning and memory in humans. Furthermore, by modulating these targets, we have restored synaptic plasticity in the conditioned courtship paradigm to wild type levels. Given that FMR1 has been implicated as interacting with so many proteins of diverse function, our optimism is tempered with caution since it would seem unlikely that just correcting one protein's function would be able to successfully rescue extremely diverse phenotypes. However, we feel that we have identified novel potential targets to attempt to modulate pharmacologically as a means to potentially restore synaptic plasticity phenotype in humans.

Experimental Procedures Drosophila Strains. A thorough explanation of the relevant genetics of the Drosophila strains used in the study can be found in Dockendorff et al, 2002. The Drosophila strains were cultured at 25° C. in 50-70% humidity in a 12 hr: 12 hr light:dark (LD) cycle on corneal-sucrose-yeast medium that was supplemented with the mold inhibitor methyl-paraben and autoclaved. In some cases additional compounds were added in the form of MPEP at 20 μg/ml, 200 μg/ml or 1,000 μg/ml, LY341495 at 400 nM, LiCl at 5 or 50 mM or NaCl at 5 or 50 mM.

Behavioral Training and Testing. Virgin male flies were collected under ether anesthesia within 4 hours of eclosion. Males were placed in individual small food tubes (15×75 mm plastic tubes containing 10-15 mm of food). The females that were used for targets were shi kept at 30 degrees, so that males would not eclose, and kept in food vials in groups of 10-15. Flies were aged for twenty days in a 12:12 LD at 25° C. before behavioral training and testing. All testing was performed during the relative light phase. Mated females were 5 days old and observed to a mated the night before training. The virgin females that were used as targets were 4 days old. Male flies were assigned to random groups and blinded training and testing was performed (Siegal and Hall, 1979, Kane et al, 1997, and McBride et al, 1999).

Histology of the Mushroom Bodies and Antennal Lobes. Staining and confocal microscopy was performed (McBride et al, 1999 and Dockendorff et al, 2002), but there were no differences found in the mushroom bodies of antennal lobes of mutant flies (Data not shown).

The effect of 200 μg/ml of MPEP on brain morphology. The antennal lobe, mushroom bodies alpha, beta and gamma lobes, and the dendrites in the MBs were also evaluated. There were no differences between the mutant and control lines in the morphology of these structures. Also, MPEP had no effect on the overall morphology of these structures (Data not shown).

Statistics. CIs of tested males were subjected to arcsin square root transformations to approximate normal distributions (McBride et al, 1999; Joiner and Griffith, 1997). ANOVAs were performed on pairwise comparisons of arcsin-transformed data to get critical p-values. All statistics were performed using Statview 3.0.

### EXAMPLE 2

### The Role of the Fragile X Protein in Drosophila melangaster in Age Related Memory Impairment and the Alleviation of this Effect by Pharmacological Treatment

### Example Summary

In Example 1, we demonstrated a requirement for functional dFMR1 protein for memory after training in Drosophila melanogaster. Here, for the first time, we examine age related cognitive decline in a Drosophila model of a human disease characterized by age related cognitive decline. We demonstrate that antagonizing the Drosophila group II metabotropic glutamate receptors (mGluRs) can prevent an age dependent deficit in learning during training in flies with no dFMR1 expression. Furthermore, we show that treatment with MPEP can continue to restore naive courtship, memory at immediate recall (0 minutes) and short-term memory (60 minutes) after training in old flies. This raises the possibility that mGluRs may be a potential target for counteracting age related memory impairment in Fragile X syndrome in humans.

One proposed explanation of the learning and memory deficits of Fragile X is altered shape and number of dendritic spines. The phenotype of abnormal dendritic spine morphology has been identified in affected humans at autopsy (Hinton et al, 1991) and is consistent with the theory that dendritic spine dysgenesis may be involved in mental retardation in humans (Purpura, 1974). Currently, there is no effective treatment to correct the cognitive deficits associated with Fragile X syndrome.

In mammals, experiments altering the expression level of FMR1 are complicated by the fact that there are two related genes, namely Fragile X related proteins (FXRP) 1 and 2, which are suspected to compensate for phenotypic deficits in the knockout mouse model (Bakker and Oostra, 2003). In Drosophila there is only one gene, dFMR1, which shares extensive amino acid homology and conservation of several key domains including KH domains, RGG box and the ribosomal association domain (Wan et al, 2000). Previously, we utilized the conditioned courtship paradigm to assess cognitive abilities in young adult Drosophila lacking dFMR1 expression. Five days of age is basically a young adult in flies, whereas 20 days is approximately old age. In young adult mutant flies, learning during training was intact, but there was no memory at 0 minutes after training (immediate recall) or 60 minutes after training (short-term memory). This memory deficit was corrected by pharmacologic treatment with MPEP (Example 1). This prompted us to look at whether there is any age related impairment of behavioral plasticity in 20 day old mutant flies, and whether the same treatment could continue to restore memory at 0 and 60 minutes after training in these flies. In order to do this, we decided to use Rescue (dFMR1-3+wild type rescue fragment) and FS (frame shift, dFMR1-3+frame shifted rescue fragment) lines from Dockendorff et al, 2002. These flies could be given different treatments prior to eclosion (development) or from eclosion until 20 days of age (adulthood), when testing occurred. Each line was divided into four groups placed on control food (CT) in development followed by CT food in adulthood, CT food in development followed by MPEP (86.6 μM) containing food (M) in adulthood, M food in development followed by M food in adulthood, or M food in development followed by CT food in adulthood. Then we tested each of these groups for naive courtship behavior towards a virgin female target, quality of that naive courtship activity, learning during training with a previously mated female, immediate recall at 0 minutes post training, short-term memory at 60 minutes post training, olfaction, visual acuity, locomotion and ability to discriminate virgin and previously mated females.

In the conditioned courtship paradigm, a male fly learns to modify his courtship behavior after experience with an unreceptive female; it is a multi-sensory paradigm involving associations from more then one sensory input (Siegel and Hall, 1979; for review see Hall, 1994). It is a complex associative learning paradigm and was utilized to assay learning and memory in this article. Courting male flies perform a characteristic sequence of behaviors: orienting toward and following the female, tapping her with his forelegs, vibrating one or both wings, licking her genitalia, and attempting copulation (Sturtevant, 1915; Bastock and Manning, 1955; Bastock, 1956). These behaviors are repeated with some variation until successful copulation occurs. Virgin females will generally respond by mating; however, recently mated females will be unreceptive to male courtship (Spieth, 1974). The naive male will find a previously mated female to have a pheromonal repertoire that is less provocative then that of a virgin female target. A naive male paired with a mated female will initially court her, but his courtship activity soon decreases; after 1 hour of experience with the mated female, his courtship when subsequently paired with a virgin female remains depressed for 2-3 hours (Siegel and Hall, 1979). These behaviors are quantified as a courtship index (CI) which is defined as the percentage of time a male fly spends performing any of the six courtship steps toward a target female in a ten minute test period. A decrease in CI during or after training with a previously mated female is indicative of behavioral plasticity in the form of learning during training or memory post training.

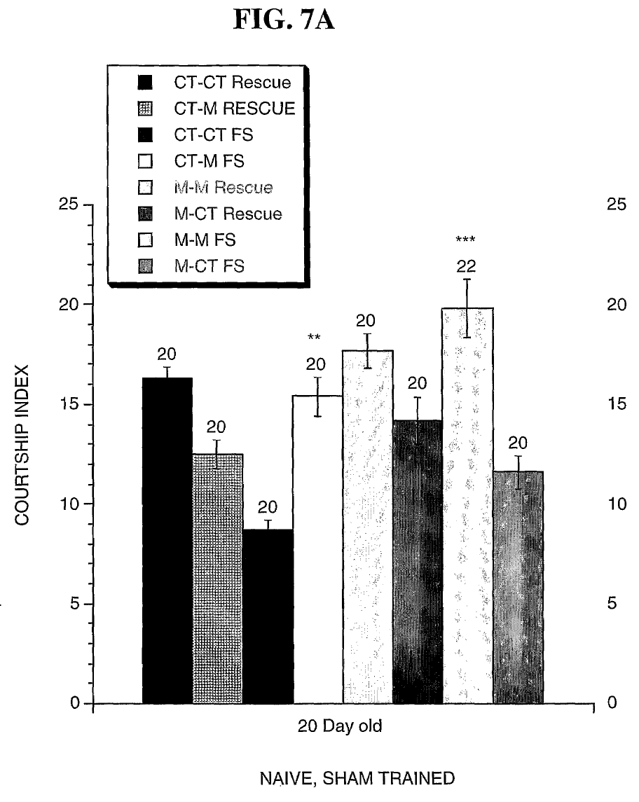

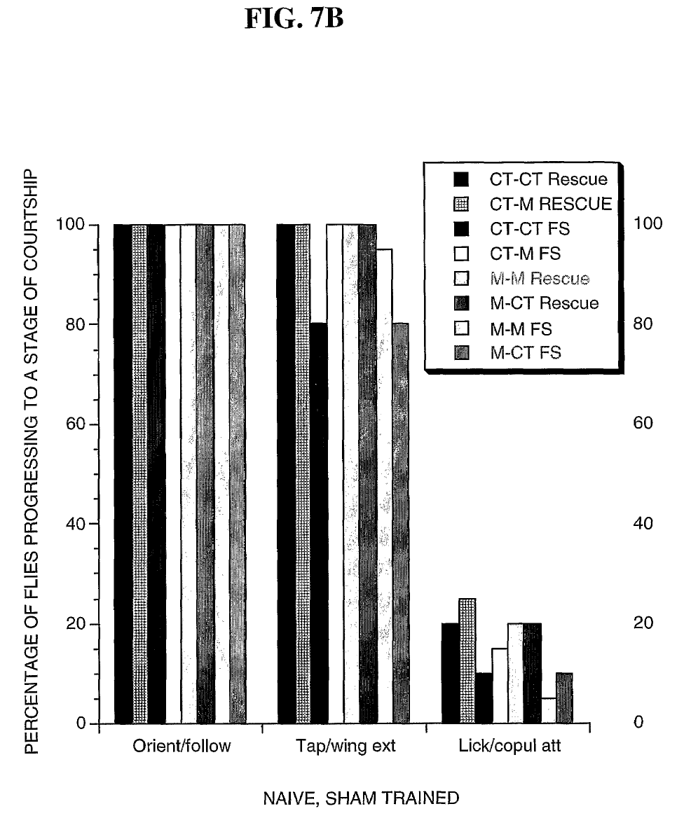

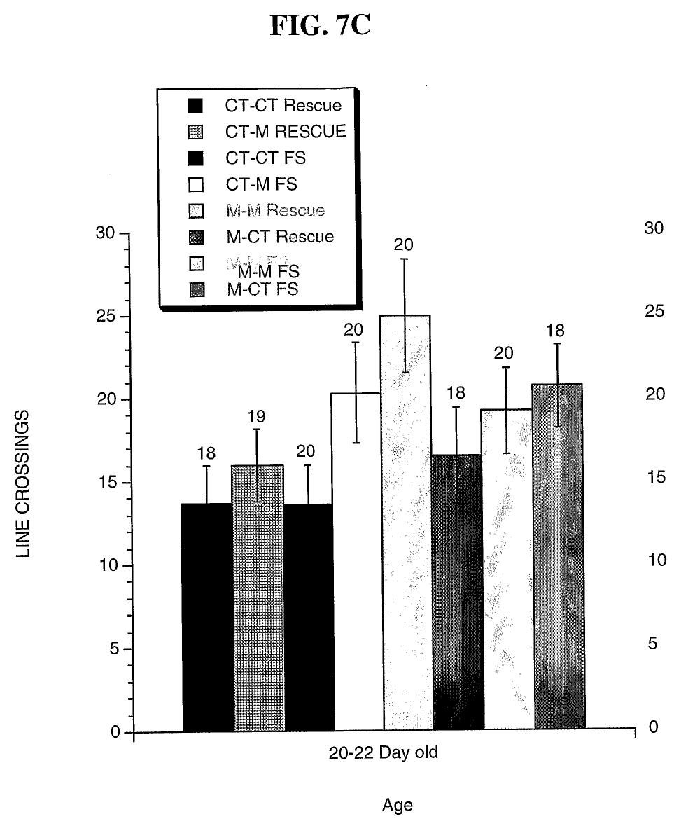

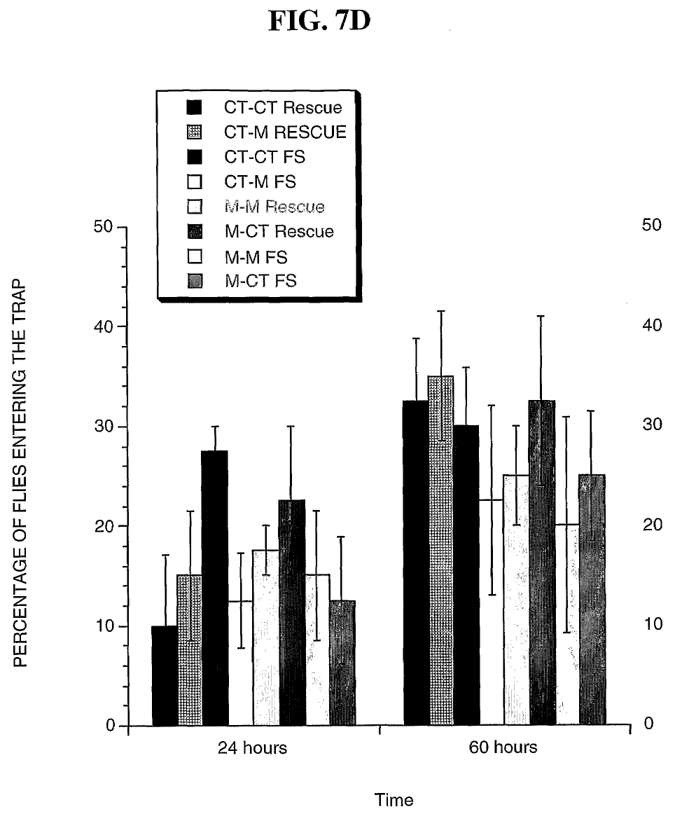

When Rescue flies were raised on CT food and then placed on M food as adults, courtship activity was depressed relative to CT-CT Rescue flies; therefore, giving this drug to healthy flies is not without some side effects (FIG. 1A). Rescue flies on M food in development and then placed on either M or CT food as adults courted as vigorously as CT-CT Rescues flies. FS flies raised on C food and then placed on M food as adults show a significant increase in courtship activity relative to CT-CT FS flies (FIG. 7A). FS flies raised on M in development and adulthood also showed a significant increase in courtship activity relative to CT-CT FS flies. Therefore, giving this drug in adulthood only is effective in enhancing naive courtship behavior. However, M-CT FS flies did not demonstrate an increase in naive courtship activity. This shows that development in the presence of M food without M food as adults is not able to restore naive courtship behavior in FS flies. Further analysis of the quality of courtship that was performed by naive males was assessed by binning the number of males to advance to a particular phase of courtship for each genotype and pharmacologic treatment (FIG. 7B). A higher percentage of the CT-CT FS and M-CT FS groups failed to advance to later stages of courtship compared to all other groups. This demonstrates that in adulthood, but not in development alone, MPEP treatment can affect the quality of naive courtship behavior in FS flies. Locomotion was assayed and found to be similar in all groups (FIG. 7C). Olfaction was also assayed and found to be similar in all groups, although significantly reduced from 5 days of age (FIG. 7D). Visual acuity was assayed and found to be similar in all groups and was also reduced from 5 days of age (FIG. 7E).

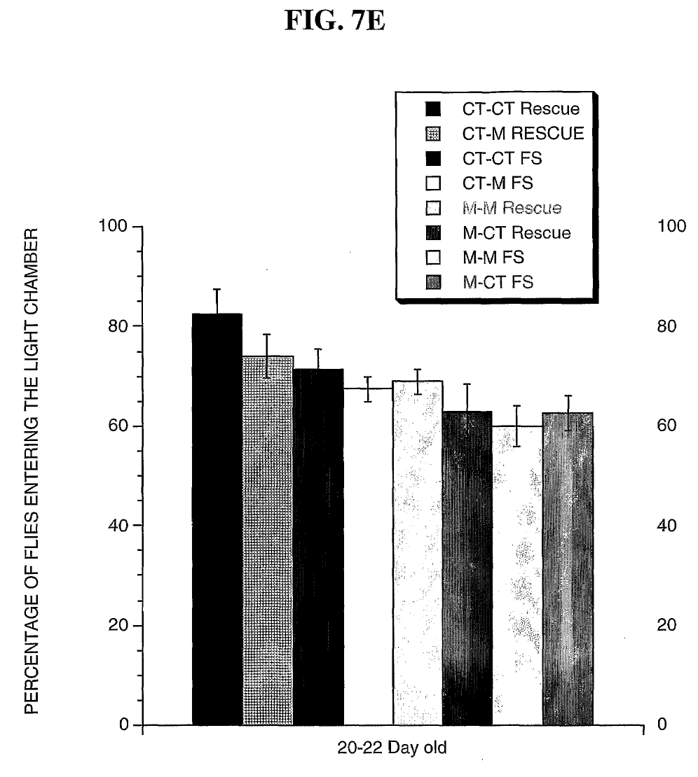

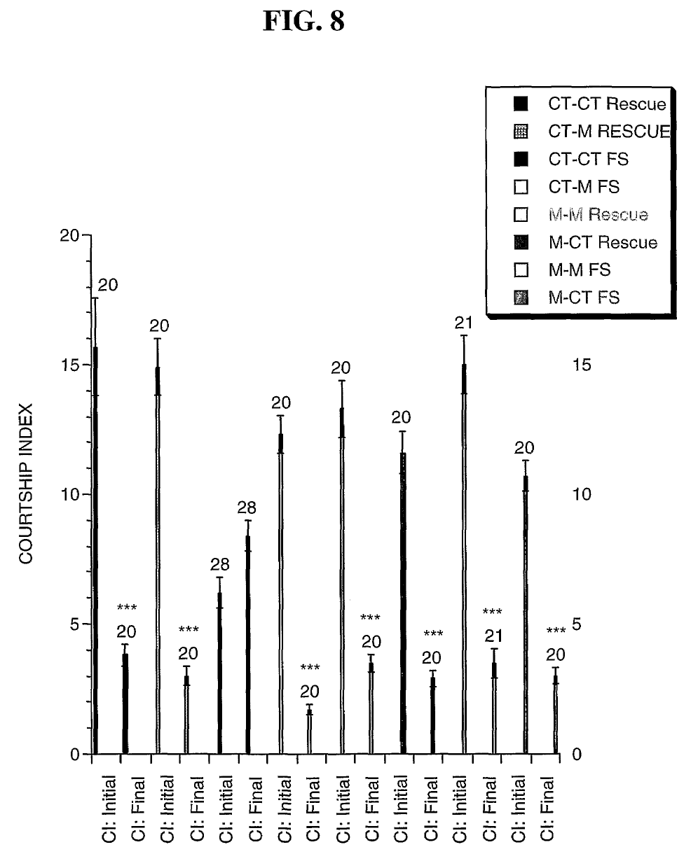

In order to assess learning during training, the male flies were placed in a training chamber with a previously mated female for one hour. The amount of time the male spent courting in the first ten-minute interval was compared to the amount of time the male spent courting the female target in the last ten-minute interval (FIG. 8). The CT-CT FS flies did not show a decrease in time spent courting during the training period, indicating no learning during training. As young adults, the CT-CT flies did show learning during the training session (p<0.0001, Example 1). The initial and final courtship levels of all other groups show significant depression from the initial to final intervals indicating that all groups demonstrated learning during training (FIG. 8). This demonstrates that treatment by MPEP in development, adulthood or both is able to restore learning during training, which is normally deficient in old FS flies grown on solely CT food.

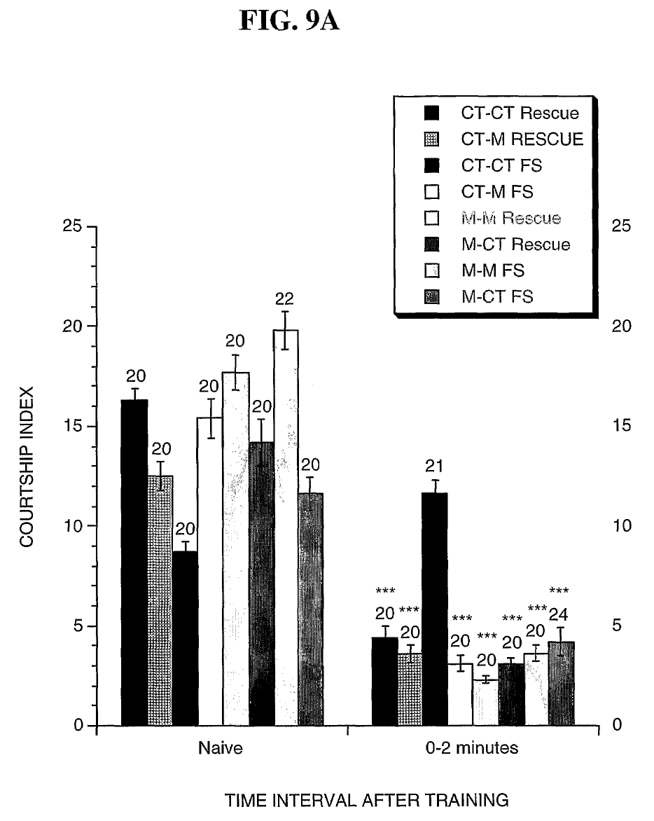

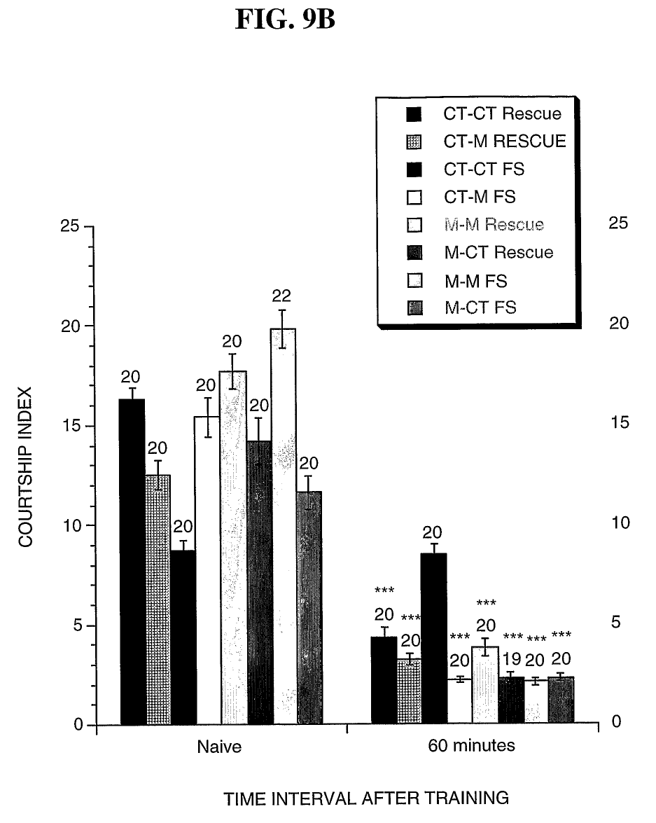

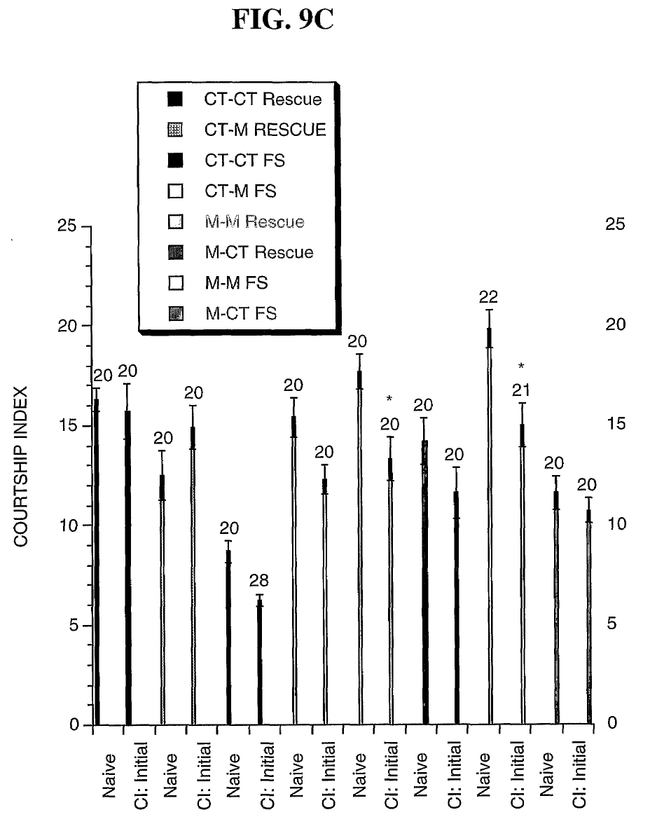

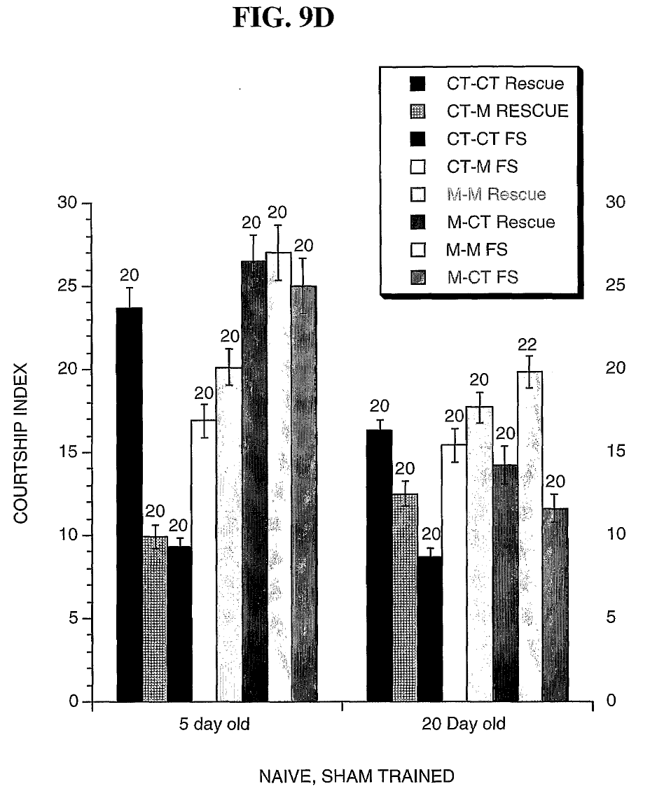

After the one training session with a previously mated female, the male is immediately placed with a virgin target female for a ten-minute interval to obtain a CI for the immediate recall time point. This is then compared to the courtship of naive males placed in the training chamber for one hour with no female, and then placed with a virgin target female for a ten-minute interval (FIG. 9A). The CT-M Rescue line shows depressed courtship activity immediately after training, indicating that M food as adults is not impairing behavioral plasticity in these flies, although it did depress naive courtship. This is also critical since this group had very similar naive courtship as the CT-CT FS flies; it shows that the behavioral plasticity of the CT-CT FS group is not missed due to an artifact of low naive courtship. CT-CT FS flies court just as vigorously immediately after training as naive CT-CT FS flies (FIG. 9A). This demonstrates that no experience dependent behavioral plasticity occurs in 20-day-old CT-CT FS flies at immediate recall, just as is seen in young adults. All Rescue groups demonstrate depression of courtship activity immediately after training relative to group matched naive flies. This indicates that M food in development or adulthood or both does not adversely affect immediate recall in Rescue groups. The remaining FS groups that were treated with MPEP, all display experience dependent reduction of courtship activity immediately after training when compared to group matched naives (FIG. 9A). Therefore treatment with MPEP in development alone, in adulthood alone, and treatment in development and adulthood together are all sufficient to restore synaptic plasticity in FS flies.

In order to examine short-term memory at 60 minutes after training, after the one hour training session with a previously mated female, the female is removed and the male is placed in a holding chamber for 60 minutes, then subsequently placed in a testing chamber with a virgin female (FIG. 9B). The CT-M Rescue line shows depressed courtship activity at 60 minutes after training, indicating that M food as adults is not impairing behavioral plasticity at 60 minutes post training in these flies, although it did depress naive courtship. CT-CT FS flies court just as vigorously at 60 minutes after training as naive CT-CT FS flies. This demonstrates the absence of short-term memory in CT-CT FS flies. The Rescue groups treated with MPEP in development alone, in adulthood alone or in both development and adulthood demonstrate depression of courtship activity at 60 minutes after training relative to group matched naive flies to the level of p<0.0001, FIG. 9B. This indicates that M food in development, adulthood or both development and adulthood does not adversely affect short-term memory in Rescue groups. The FS groups that were treated with MPEP in development alone, adulthood alone or in both development and adulthood display experience dependent reduction of courtship activity at 60 minutes after training when compared to group matched naives M-M FS. Therefore short-term memory is also rescued in 20-day-old FS flies by MPEP treatment, as it is in young FS flies. Lithium, Ly341495, MPPG and MTPG also rescue short-term memory in 20-day-old FS flies.

Flies were also tested for ability to discriminate between virgin and previously mated females in FIG. 9C. Normally a young adult naive male displays less courtship when paired with a previously mated female target compared to when paired with a virgin female target. However, as 20-day-old flies only the M-M Rescue and M-M FS flies displayed this ability (FIG. 9C). This phenomenon, however, does not seem to be critical for behavioral plasticity at 0 minutes or 60 minutes after training since all of the other groups except CT-CT FS had intact behavioral plasticity without displaying a significant difference in this assay.

In Drosophila there are five phases of memory as have been dissected out by several genetic and pharmacological studies (Greenspan, 1995). Depending on when the fly is assayed there is an immediate recall at 0-2 minutes post training; short-term memory out to 1 hour; medium-term memory out to 6 hours; anesthesia resistant memory out to two days; and long-term memory which lasts up to 9 days post training and appears to be protein synthesis dependent (Tully et al, 1994; Yin et al, 1994; Yin et al, 1995). In the conditioned courtship paradigm, learning during training can be assayed by comparing the decrease in CI during the first ten minutes after the male is paired with an unreceptive female with the CI of the last ten-minute period of the pairing. Flies typically show a 40% or more decrease in courtship activity (Joyner and Griffith, 1997; Kane et al, 1997).

As young adults, flies lacking functional dFMR1 expression display deficits in immediate recall and short-term memory. However, as older flies they display an additional deficit in learning during training, which is intact at 5 days of age. This is an age dependent decline in cognitive ability that is analogous the to what happens to humans afflicted with Fragile X syndrome. In Drosophila, age related memory impairment was seen in an altered version of the conditioned courtship paradigm (where male flies were trained with previously mated females and then tested for immediate recall with previously mated females) in flies with mutations in the kynurenine pathway (Savvateeva et al, 2000). Additionally, in the olfactory association paradigm, a deficit in medium-term memory was found in wild type flies, and this was shown to be do to alterations in the amnesiac protein expression that occur with aging (Tamura et al, 2003). Since the histology does not indicate cell death as the cause of cognitive dysfunction in young or old FS flies, we suspect that the problem is due to synaptic silencing in living neurons, where the synapses are no longer functioning properly.

The activation mGluRs, of an uncharacterized subtype, by the addition of exogenous glutamate has been shown to induce an intracellular calcium rise and PKC activation dependent form of long-term depression (LTD) leading to the synthesis of FMR1 protein in mice (Weiler and Greenough, 1999). FMR1 knockout mice have enhanced LTD in the hippocampus as a result of increased group I subtype 5 mGluR activity (Huber et al, 2002). In the cortex of knockout mice, long-term potentiation (LTP) was shown to be significantly reduced (Li et al, 2002). In Example 1 we demonstrated that rebalancing LTD vs LTP by treating with MPEP could counteract this memory deficit in young adult FS flies. MPEP in mammals is a selective non-competitive antagonist of subtype 5 group I mGluRs. Drosophila only posses group II mGluRs, but the binding pocket for MPEP is nonetheless conserved. By antagonizing mGluRs, we are increasing adenylate cyclase activity to increase LTP and decreasing group II mGluR induction of LTD in a manner dependent on PLC and IP3R activity (Huang et al, 1999b; Otani et al, 2002). In this paper we demonstrate that treatment with MPEP in development or adulthood is sufficient to rescue memory at immediate recall and short-term memory in FS flies into old age. This is an important finding because just rescuing memory as young adults left open the possibility that the effectiveness of the MPEP may where off over time. Only treatment with MPEP in adulthood was able to significantly increase naive courtship behavior. The age dependent phenotype, which is having impaired learning during training, was prevented when FS flies were treated with MPEP in either development, adulthood, or both. Therefore antagonizing group II mGluRs may be a potential therapeutic target for prolonged correction of the cognitive deficits associated with Fragile X syndrome as well as the progressive cognitive decline that it entails. Additionally, the strategy of modulating the activity of group II mGluRs to achieve a rebalancing of LTD vs. LTP to prevent synaptic silencing, may be a strategy that is generally applicable to the treatment of other diseases involving progressive cognitive decline such as Alzheimer's disease, tauopathies and Huntington's disease.

### Experimental Procedures

Drosophila Strains. A thorough explanation of the relevant genetics of the Drosophila strains used in the study can be found in Dockendorff et al, 2002. The Drosophila strains were cultured at 25° C. in 50-70% humidity in a 12 hr: 12 hr light:dark (LD) cycle on corneal-sucrose-yeast medium that was supplemented with the mold inhibitor methyl-paraben and autoclaved. In some cases MPEP was added at 200 μg/ml or 1,000 μg/ml.

Behavioral Training and Testing. Virgin male flies were collected under ether anesthesia within 4 hours of eclosion. Males were placed in individual small food tubes (15×75 mm plastic tubes containing 10-15 mm of food). The females that were used for targets were shi kept at 30 degrees, so that males would not eclose, and kept in food vials in groups of 10-15. Flies were aged for twenty days in a 12:12 LD at 25° C. before behavioral training and testing. All testing was performed during the relative light phase. Mated females were 5 days old and observed to a mated the night before training. The virgin females that were used as targets were 4 days old. Male flies were assigned to random groups and blinded training and testing was performed (Siegal and Hall, 1979, Kane et al, 1997, and McBride et al, 1999).

Statistics. CIs of tested males were subjected to arcsin square root transformations to approximate normal distributions (McBride et al, 1999; Joiner and Griffith, 1997). ANOVAs were performed on pairwise comparisons of arcsin-transformed data to get critical p-values. All statistics were performed using Statview 3.0.

### EXAMPLE 3

### The Effect of MPEP on Learning and Memory in a Mutant Presenilin Drosophila Model of Alzheimer's Disease

Using methods similar to those described in Examples 1 and 2, we evaluated the effect of MPEP on various aspects of learning and memory in a Drosophila model of Alzheimer's disease with reduced expression of presenilin.

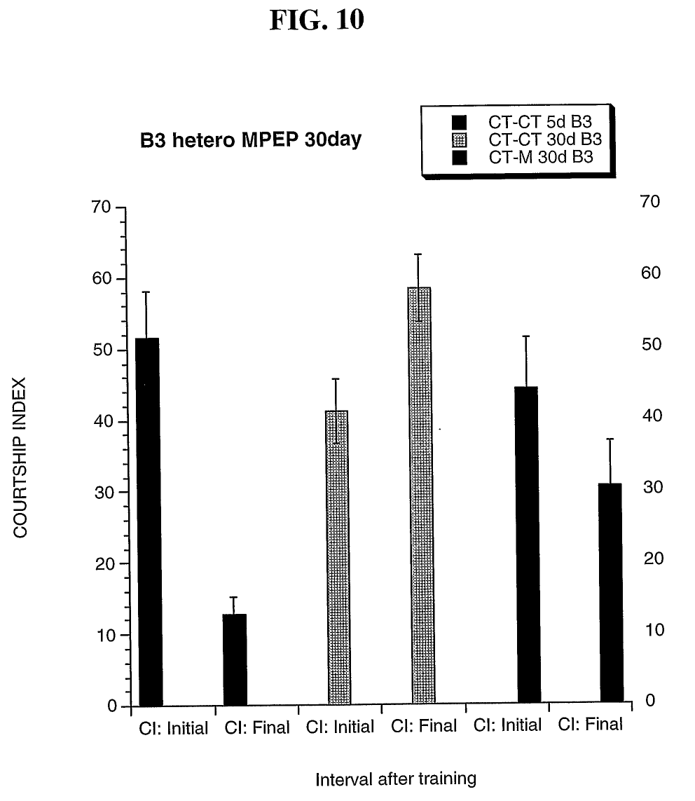

The results are summarized in FIG. 10. Flies in all three groups are heterozygous for the Drosophila presenilin gene. Each group has one wild type presenilin gene and one null presenilin gene, resulting from a deletion at the presenilin locus or a mutation at the presenilin locus. Learning during training was assayed in flies kept on control food until tested at five days of age, kept on control food until tested at 30 days of age or kept on control food for the first five days after eclosion then switched to food containing 200 μg/ml of MPEP until being moved back to control food the day before being tested at 30 days of age. As shown in FIG. 10, flies heterozygous for the presenilin mutation have normal learning during training at 5 days of age p<0.005, but impaired learning during training at 30 days of age. This age-dependent deficit in learning during training is apparent in ten mutant presenilin Drosophila lines evaluated. In flies treated with MPEP, learning during training was restored at 30 days of age (p<0.05). Therefore, this Drosophila model of Alzheimer's disease displayed age-dependent memory impairment that is rescued by treatment with MPEP.

We have also discovered that there is a deficit in long term memory in these flies as young adults, and there is also an age dependent deficit in these flies in short term memory at 30 days of age (data not shown). This age dependent deficit in short term memory is rescued by treatment with LY341495. Additionally, this age dependent deficit is rescued by lowering IP3R expression by making these presenilin mutant lines heterozygous for an IP3R deletion (data not shown).

Familial Alzheimer's disease-linked presenilin mutations generally result in increased Abeta 42 production, often without an overall increase in Abeta levels. This has often been cited as evidence of a pathogenic gain of toxic function mechanism. However, evidence has continued to accumulate indicating that the pathogenic mechanism may really be a loss of presenilin function, at least in some cases. Additionally, several lines of evidence point to an enhancement of LTD and or decrease in LTP in animal models of Alzheimer's disease, just as is the case for the Fragile X knockout mice. Lowered presenilin expression has been shown to be able to cause impaired synaptic plasticity and later in life neurodegeneration in mice with transgenically lowered presenilin expression (Saura et al, 2004). This is analogous to our Drosophila model of Alzheimer's disease, where we used a lowered presenilin gene dosage, presumably leading to a lowered presenilin protein level, to examine the phenotype of age dependent cognitive decline. Therefore, we believe that treatment with the type two mGluR antagonist is a viable therapeutic approach to rebalancing LTD/LTP in Alzheimer's disease to restore cognitive abilities in humans.

### EXAMPLE 4

### Further Studies with mGluR Antagonists

Methods utilized in this example are described in Examples 1-3.

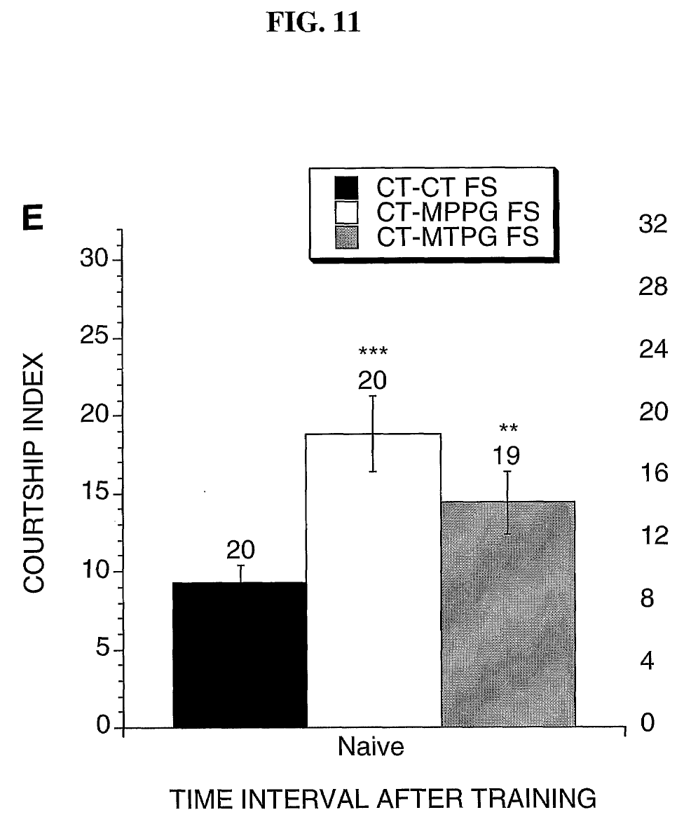

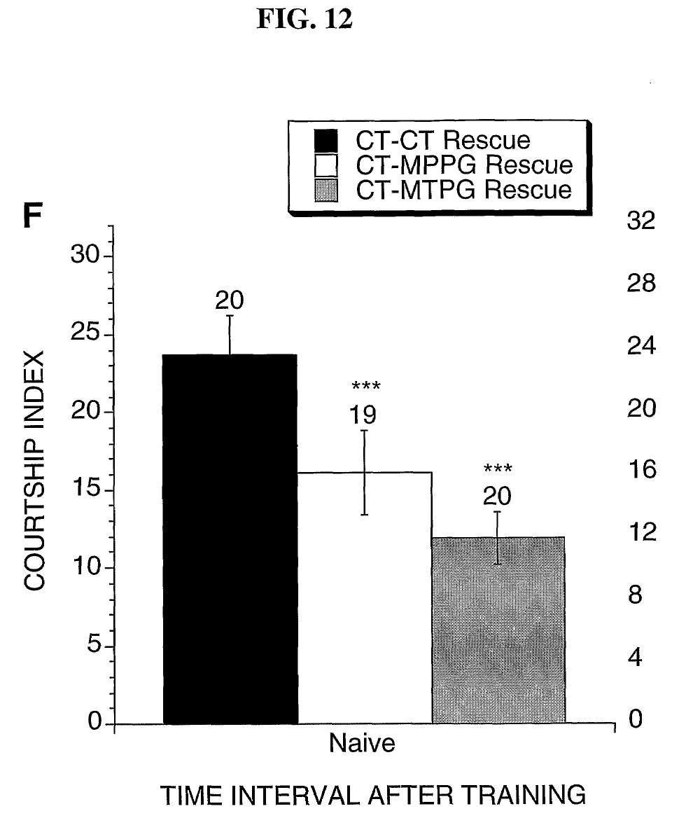

The naive courtship levels of flies lacking dfmr1 activity and treated with low doses of MPEP, LY341495 and LiCl. Adult FS and Rescue flies were treated with 573 mM MPPG or 348 μM MTPG. This treatment resulted in a significant increase in courtship activity for the FS flies (p<0.0001 and p<0.005) (FIG. 11) but a significant decrease in courtship activity (p<0.0001, for both) in the Rescue flies (FIG. 12).

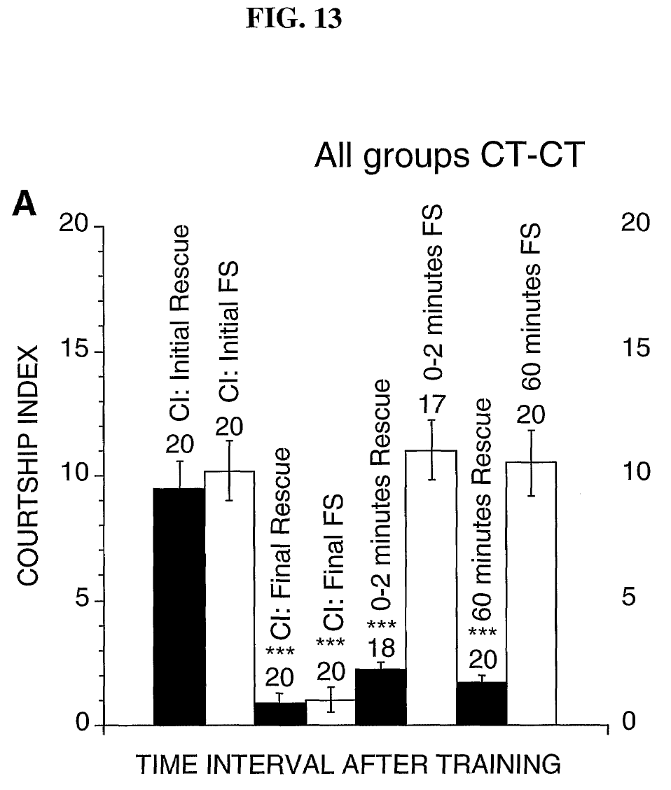

Effects of drug treatment on short-term memory (60 mins) in flies lacking dfmr1 activity with mated female targets. Short-term (60 min.) memory was measured in Rescue and FS flies that were either fed control food or underwent various drug treatments as described below. Short-term memory was measured by placing a trained male in a holding chamber for 60 minutes (after being trained for one hour with a previously mated female), then subsequently placing him in a testing chamber with a mated female target for a ten-minute courtship interval (C.I.). This C.I. was compared to the C.I. obtained for naïve courtship of a previously mated female, i.e., C.I. during the first 10 minutes of the training session with a previously mated female. Additionally, for reference the C.I. during the last 10 minutes of the training period was also determined. Results are shown in FIGS. 13-17. Numbers of flies are indicated above each bar for all groups. The levels of significance are indicated as follows; *=p<0.05; **=p<0.005; ***=p<0.0001.

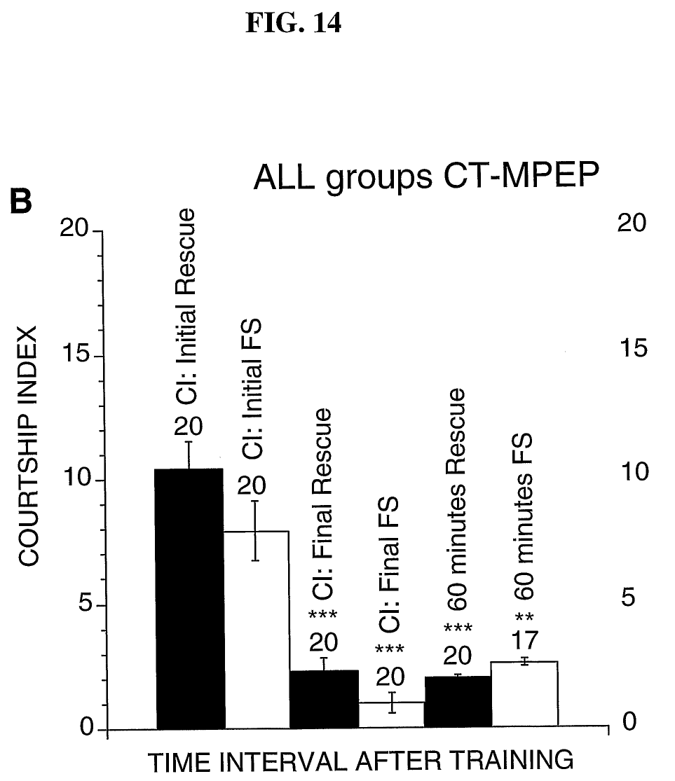

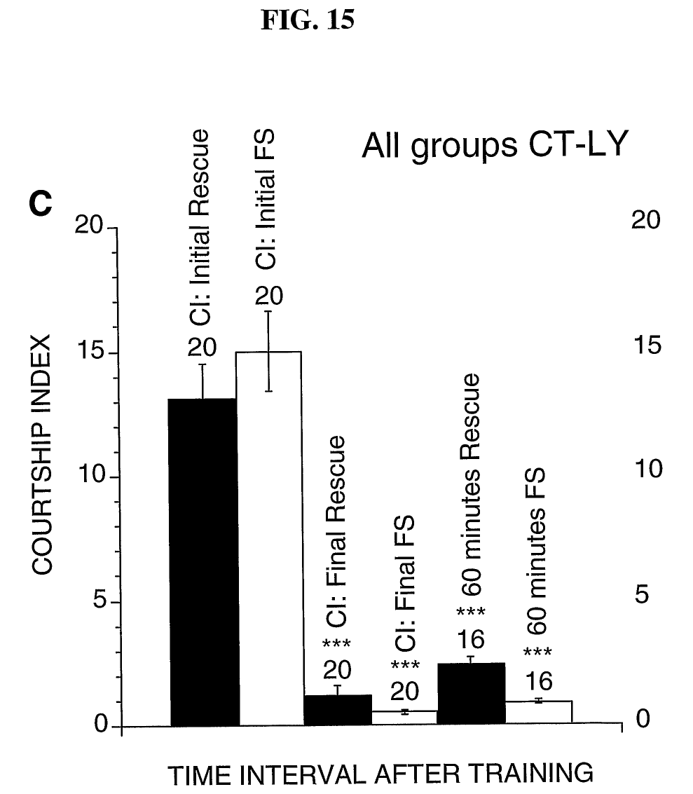

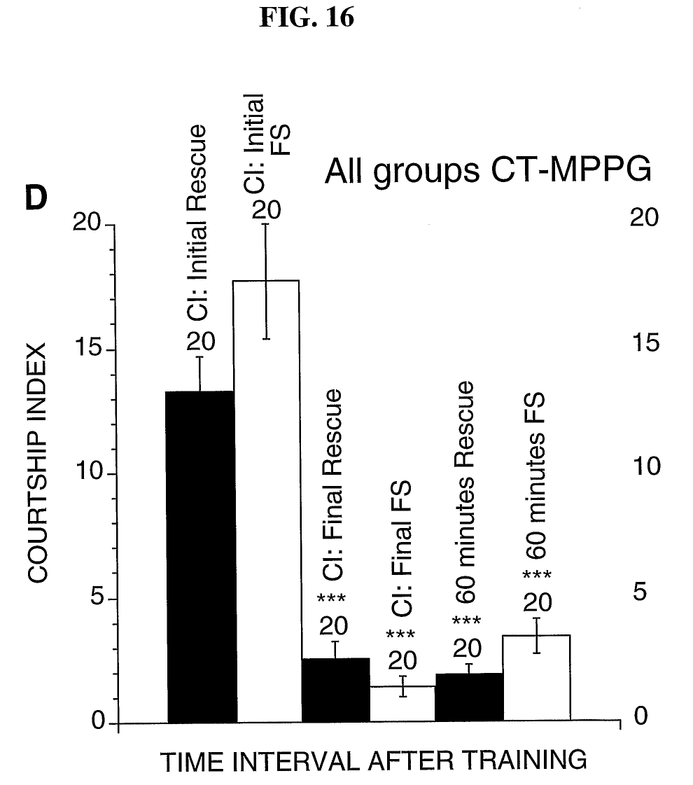

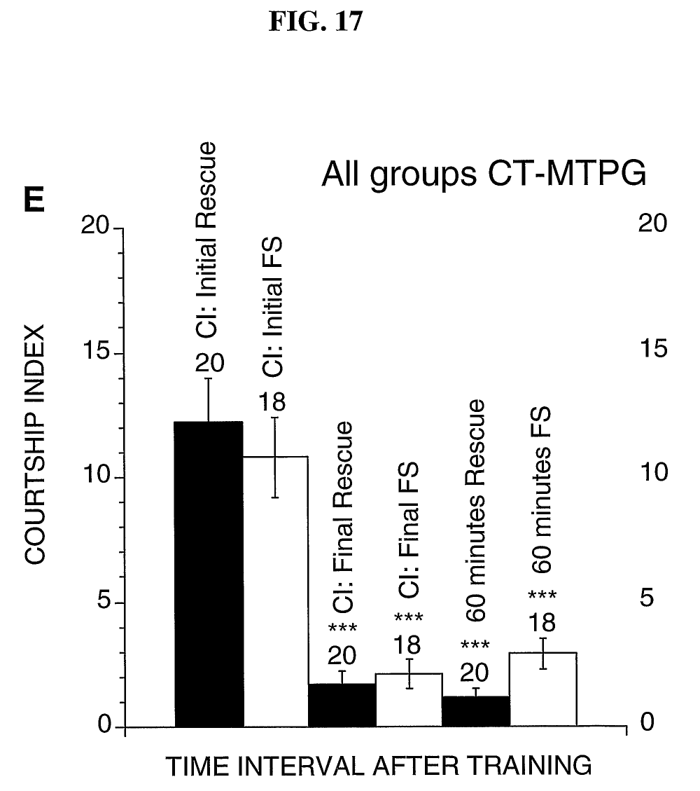

Rescue flies kept on only control food demonstrated memory at 0-2 minutes and 60 minutes after training. In contrast, FS flies kept on only control food did not show memory at either time point (FIG. 13). Rescue and FS flies treated with 8.6 mM MPEP demonstrate memory at 60 minutes post training (FIG. 14). Rescue and FS flies treated with 400 nM LY341495 demonstrate memory at 60 minutes post training (FIG. 15). Rescue and FS flies treated with 573 mM MPPG demonstrate memory at 60 minutes post training (FIG. 16). Rescue and FS flies treated with 348 mM MTPG demonstrate memory at 60 minutes post training (FIG. 17).

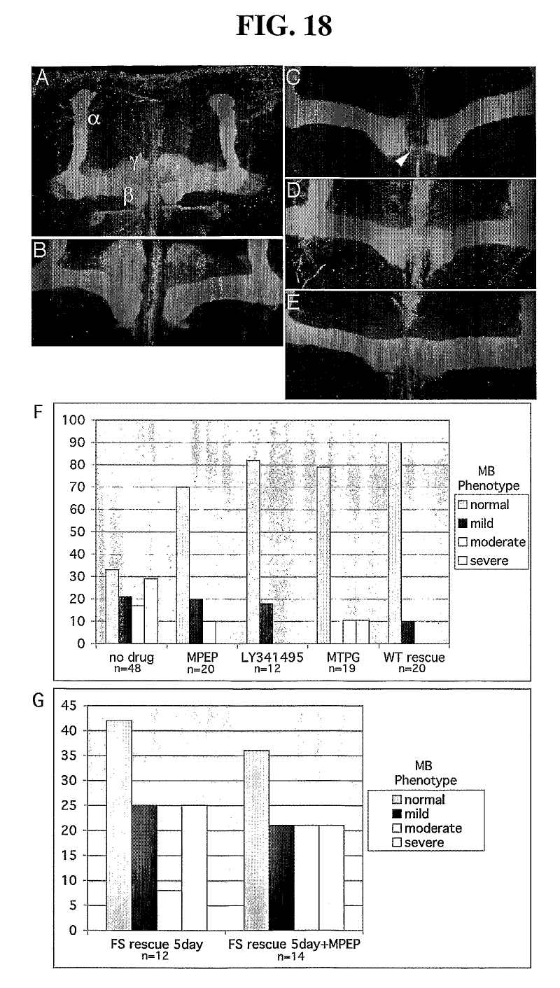

Histological analysis was performed on relevant experimental flies, showing the fusion of MB beta lobes and the rescue of these fusions with in mGluR antagonists (FIG. 18). Brains from (0-1 day old) dfmr1 mutant adult flies were stained with anti-fasciclin II (ID4) and a rhodamine-coupled secondary antibody. The α, β, & γ lobes of the MBs are clearly labeled with this antibody and appear normal in this mutant brain (Panel A). Panels B-E show a higher magnification of the β-lobes at the midline. Panel B shows a dfmr1 mutant brain with normal β-lobes. Panels C-E show mutants brains displaying a C) “mild” (arrowhead), D) “moderate” and E) “severe” level of midline crossing by the α-lobes. Panel F shows experimental results revealing the penetrance of the β-lobe fusion detected in untreated “no drug” (0-1 day old) dfmr1 mutant brains, or those fed food containing 8.6 mM MPEP, 400 nM LY341495, 348 mM MTPG. WT rescue flies are dfmr1 mutants containing one copy of the dfmr1 genomic rescue fragment. The number of brains examined are listed below each group. Panel G shows dfmr1 mutant brains from 5 day old adults that were either fed control food the entire time (FS rescue 5 day) or were fed food containing 8.6 mM MPEP for five days starting immediately after eclosion.

### Discussion

Restoration of naive courtship with mGluR antagonists. The CT-MPPG and CT-MTPG FS flies displayed significant increases in courtship (p<0.0001 and p<0.005, FIG. 11). The CT-MPPG and CT-MTPG Rescue flies displayed significant decreases in courtship (p<0.0001 and p<0.0001)(FIG. 12). Taken alone, the results from each individual treatment provide some evidence for specificity of the target. However, when it is considered that all five of the treatments give a common result, a very strong case is made is consistent with the model that reduction of mGluR activity restores the naïve courtship activity levels of the dfmr1 mutant flies.

Restoration of short-term memory with mGluR antagonists. To establish that the failure to observe memory was not due to a problem with recognizing or processing the appropriate cues from the virgin female target, we used a modified version of the conditioned courtship paradigm where the male is paired with a mated female target subsequent to training (Kane et al., 1997; Joiner and Griffith, 1997; Joiner and Griffith, 1999; Kamashev et al., 1999). Again, with pharmacologic treatment it is necessary to utilize more then one compound to confirm the specificity of the target, so in this case we used four mGluR antagonists. CT-CT Rescue flies demonstrate memory at immediate recall and short-term memory, whereas CT-CT FS flies fail to demonstrate memory of training at either time point (FIG. 13). Rescue flies treated with 86 μM MPEP, 400 nM LY341495, 573 μM MPPG and 348 μM MTPG demonstrated intact short-term memory (FIGS. 14-17). Additionally, FS flies treated with 86 μM MPEP, 400 nM LY341495, 573 μM MPPG and 348 μM MTPG demonstrated restoration of short-term memory (FIG. 14-17). This shows that the memory deficit observed in mutant flies is not due to a sensory processing impairment, but is definitively a memory impairment. It is also important to note that the robust deficit in synaptic plasticity in FS flies, with regard to memory, is a critical extension of the previous models of Fragile X syndrome in model organisms, since this is one of the most prominent aspects of the human disorder. Furthermore, we have shown that antagonism of the mGluRs can restore synaptic plasticity in the Drosophila model of Fragile X syndrome.

Rescue of β-lobe fusions with mGluR antagonists. Previously it has been established that the mushroom bodies are involved in learning and memory in the conditioned courtship and the odor shock classical conditioning paradigms in Drosophila (deBelle and Heisenberg, 1994; Joiner and Griffith, 1999; McBride et al, 1999; Zars et al, 2000; Paschel and Preat, 2001). Recent studies of dfmr1 mutants have revealed that the β-lobes of the mushroom bodies (MB) cross over the midline and fuse at a fairly high frequency (Michel et al., 2004; Pan et al. 2004). Since such defects have been found in the mutant linotte, which also displays memory defects (Moreau-Fauvarque et al., 1998; Simon et al., 1998), we investigated whether we could observe this defect in our mutants and if it was rescued by treatment with mGluR antagonists. When labeled with anti-FasII, we observed a range of β-lobe fusion defects in brains derived from 0-2 day old FS mutant flies, but not Rescue flies (FIG. 18). Using the scoring method described by Michel et al., 2004, we observed defects ranging from mild to severe in roughly 70% of the FS mutant brains, whereas only 10% of Rescue fly brains displayed defects and these were all mild defects (FIG. 18).

Since treatment of FS mutant flies with mGluR antagonists rescued the memory defects observed in these flies, we examined the effect of these treatments on the β-lobe fusion defect to determine if the rescue of the two phenotypes was correlated. We raised FS mutant flies in food containing 8.6 mM MPEP, 400 nM LY341495 or 350 mM MTPG, then examined the morphology of the MBs in 0-2 day old adults. With all of these drug treatments we observed rescue of the β-lobe fusion defects (FIG. 18F). For example, mild fusion defects were only observed in 18% of FS mutant brains raised in food containing 400 nM LY341495 (FIG. 18F). No effect was observed when these drugs were fed to Rescue flies during development (not shown).

This rescue of the β-lobe fusion defect suggests that prevention of this defect is key to rescuing the memory defects observed in this mutant. If this is true, then we would expect that the behavioral rescue obtained by treating FS mutant flies these drugs during adulthood alone would lead to a similar morphological rescue. To test this hypothesis, we treated FS mutant flies with 8.6 mM MPEP for four days starting at eclosion, and then transferred them to normal food for 24 hours before examining the morphology of their MBs. For comparison we also examined the MBs of flies left in control food for five days. Contrary to the results obtained when the drug treatments were performed during development, we did not observe any rescue of the β-lobe fusion defects with the treatment during adulthood (FIG. 18G). Thus it appears that rescue of this morphological defect is not absolutely necessary for the rescue of the memory defects observed in the dfmr1 mutant flies.

The data presented in this Example provides evidence that group III mGluR antagonists can restore several phenotypes in learning and memory diseases. The mGluR family in mammals is divided into 3 subfamilies (groups I, II and III) based on pharmacology, which also matches with the later determined sequence homologies. By sequence homology the group I mGluR1 and mGluR5 are most closely related to each other, while the group II (mGluR2 and mGluR3) and group III (mGluR4, mGluR6, mGluR7 and mGluR8) receptors are most closely related to one another. Indeed, the conservation is so strong between group II and group III receptors that most agonists and antagonists that are currently available that have an affinity for one group, will also modulate the at least one member of the other group often at a similar concentration, even though the affinity of such compounds for the group I receptor are much different.

In our Drosophila model, we are antagonizing the DmGluRA and possibly the DmGluRB (DmGluRX). DmGluRA has been shown to activate Gi alpha signaling, respond to some compound that modulate mammalian group II receptors and was classified as a group II mGluR (Pommier et al, 1996). However, at the concentrations used, the agonists and antagonists could also modulate the activity of mammalian group III receptors, although they would not affect group I receptors at these concentrations. Therefore, in this regard, the pharmacology used can rule out the relation of the DmGluRA to group I receptors, but not to group III receptors. Furthermore, by looking at the similarity of the primary amino acid sequence by identity and conservation, both DmGluRA and DmGluRB are nearly equally related to group II and group III mammalian mGluRs. This is illustrated by the following table.

TABLE 1
Amino acid sequence comparisons of DmGluRA and
DmGluRB to mammalian mGluRs.

| | | | |
|---|---|---|---|
| Isoform | Group | Identity | Conservation |

DmGluRA, Mammalian receptor

| | | | |
|---|---|---|---|
| 3 | (II) | 47% | 62% |
| 2 | (II) | 47% | 63% |
| 7 | (III) | 45% | 59% |
| 8 | (III) | 45% | 59% |
| 4 | (III) | 44% | 61% |
| 6 | (III) | 43% | 59% |

DmGluRB, Mammalian receptor

| | | | |
|---|---|---|---|
| 8 | (III) | 42% | 62% |
| 7 | (III) | 41% | 61% |
| 4 | (III) | 42% | 61% |
| 6 | (III) | 42% | 60% |
| 2 | (II) | 42% | 59% |
| 3 | (II) | 41% | 58% |

In our experiments we have utilized 5 compounds to show that antagonizing mGluR signaling restores the phenotypes of memory in Fragile X model flies. The MPEP molecule and lithium would clearly have effects regardless of the whether it is a group II of group III mGluR that is critical for this phenotype in Drosophila. Additionally, we have used LY341495, MPPG, and MTPG. Again, at the concentrations used, all of these compounds have activity against the group III mGluRs as well as the group II mGluRs, but not against the group I mGluRs. LY341495 at the concentration of 400 nM, which was used in this study, is a competitive antagonist of the group II (mGluR2 and mGluR3) and group III (mGluR8) receptor (Fitzjohn et al., 1998; Johnson et al., 1999; Kingston et al., 1998; Ornstein et al., 1998). MPPG and MTPG also each antagonize both the group II and group III mammalian mGluRs at the concentrations used in our study (Bushell et al, 1996; Jane et al, 1995; Huang et al, 1997; Naples and Hampson, 2001; Folbergrova et al, 2001).

Previous studies have established that group I mGluRs in mammals activate the Gq pathway, while the group II and group III mGluRs activate the Gi signaling pathway. However, there is accumulating evidence that in mammals group II mGluRs may activate the Gq signaling pathway and induce LTD in a manner dependent on PLC and InsP3R activity (Huang et al., 1997; Huang et al., 1999a; Huang et al., 1999b; Otani et al., 1999; Otani et al., 2002), and group I mGluRs are capable of activating Gi (Kriebich et al., 2004). We are outlining in this model, the possible actions of the group II mGluRs in Drosophila. To date, only Gi activity has been demonstrated to be activated by the Drosophila mGluRs; we have speculated the Gq pathway may also be activated to some degree, since it happens in mammals. Upon activation, Gq activates PLC, which produces DAG and InsP3, which can activate PKC and activate the InsP3R to release calcium from the endoplasmic reticulum. The InsP3R is involved in the modulation of cytoplasmic free calcium concentration which plays a role in intracellular signaling that regulates a diverse set of physiologic processes in cells (Berridge et al., 1989; Berridge, 1993; Mak et al., 1998). The type 1 InsP3R is involved in the establishment of LTD in the cerebellular neurons, and in the suppression of LTP in the hippocampus (Khodakhah and Armstrong, 1997; Inoue et al., 1998; Fujii et al., 2000; Nishiyama et al., 2000). Gi alpha activation inhibits adenylate cyclase (AC), thereby preventing an increase in cAMP, which prevents the activation of CREB by PKA, and thereby reduces LTP. In fact, altering the activity of CREB can enhance memory in Drosophila and additionally enhance long-term facilitation in Aplysia and LTP in mice (Yin et al., 1994; Yin et al., 1995; Roman and Davis, 2001; Vitolo et al., 2002; Bozon et al., 2003; Chen et al., 2003; Tully et al., 2003). Furthermore, it has been shown that the beta and gamma subunits of Gi can stimulate calcium release from the endoplasmic reticulum by directly activating the InsP3R (Zeng et al., 2003); thus it may also have a role in the establishment of LTD. MPEP is a group I mGluR5 non-competitive antagonist, which we use at high concentrations to block the Drosophila group II mGluR. LY341495 is a competitive antagonist of the mammalian group II mGluRs at the concentrations we used to inhibit the activity of the Drosophila group II mGluR, as are MPPG and MTPG. LiCl has many activities in cells, one of which is the lowering of InsP3 levels by inhibiting inositol monophosphatase and inositol polyphosphatase, thereby decreasing the synthesis of InsP3, as well decreasing the rate at which it is recycled (Berridge et al., 1989; Berridge, 1993; Takei et al., 1998; Williams et al, 2003). Additionally, LiCl treatment has been shown to enhance LTP in mice, and facilitate CREB activation in cultured cells (Son et al., 2003; Bullock and Habener, 1998; Grimes and Jope, 2001; Mai et al 2002). Finally, we used LiCl at concentrations of 5 mM and 50 mM. Previously, LiCl has been shown to inhibit GSK-3 alpha and beta activity and was shown to enhance LTP, facilitate CREB DNA binding activity at least partially via inhibition of GSK-3 alpha and beta, (Berridge, 1993; Berridge et al., 1989; Grimes and Jope, 2001; Mai et al, 2002; Takei et al., 1998; Williams et al, 2003). It is through the aforementioned activities that these drugs apparently decrease the establishment of LTD and increase the establishment of LTP.

### EXAMPLE 5

### Phosphodiesterase-4 Inhibition for Diseases Affecting Learning and Memory

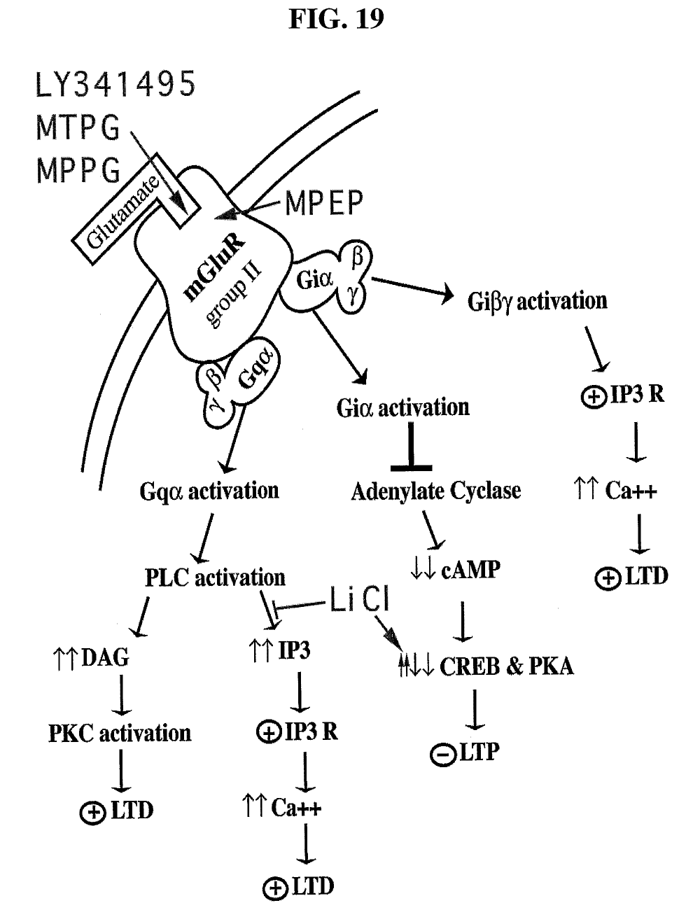

As shown in FIG. 19, which outlines the relevant signal transduction pathways, treatment with lithium, mGluR group II antagonists and mGluR group III antagonists result in increased CREB mediated gene transcription. This led us to explore the possibility that enhancing levels of cAMP may be beneficial in our Fragile X mutant flies. We treated FS and Rescue flies with 100 μM 4-[3-(Cyclopentyl)-4-methoxyphenyl]-2-pyrrolidinone (rolipram), which is a phosphodiesterase-4 (PDE-4) inhibitor (Alarcon et al, 2004; Vitolo et al, 2002; Guan et al, 2002; Bourtchouladze et al, 2003; Tully et al, 2003). 40 μM rolipram increases levels of cAMP and PKA activity when fed to Drosophila (Hou et al, 2004). PDE-4 inhibition should lead to increased levels of cAMP, thereby increasing PKA activity and leading to increased CREB mediated gene transcription. It should be noted that one of the effects of lithium is also to increase CREB mediated gene transcription. Rolipram depressed the naive courtship of both FS and Rescue flies (Table 2). However, rolipram restored short-term memory in FS flies when tested with a previously mated female target, 13.3±1.4 vs 2.7±0.6, p<0.05.

TABLE 2
Effect of rolipram on 5 day old Drosophila

| genotype | | std error | (n) |
|---|---|---|---|
| | Cin | | |
| FS | 13.3 | 1.4 | (20) |
| Rescue | 15.4 | 2.0 | (19) |
| | Cfin | | |
| FS | 2.2 | 0.5 | (20) |
| Rescue | 2.8 | 0.7 | (19) |
| | STM with PM | | |
| FS | 2.7 | 0.6 | (20) |
| Rescue | 5.5 | 1.0 | (19) |
| | Naive | | |
| FS | 3.5 | 0.7 | (18) |
| Rescue | 6.2 | 1.0 | (19) |

Additionally, mutant huntington protein disrupts CREB mediated transcription of several genes including BDNF which has been implicated in long term potentiation and memory. BDNF is involved in LTP formation. BDNF increases activity and release of tPA, tPA cleaves BDNF to mBDNF which binds TrkB causing LTP, how, well TrkB activation inhibits gsk3b activity, since gsk3b phosphorylates and CREB at residue 129 and inhibits CREB transcription, relieving this inhibition will increase CREB activity promoting LTP. Additionally, CREB can upregulate transcription of BDNF. mGluR group II/III antagonists will increase cAMP levels, increasing PKA activity which phosphorylates CREB at residue 133, increasing CREB transcription and promoting LTP, also increasing BDNF levels. Lithium also upregulates BDNF levels, enhances LTP and increases CREB transcription by inhibiting gsk3b, i.e., the actions of lithium and mGluR group II/III antagonists are both to promote LTP, decreasing the tendency of LTD to occur. It is important to keep in mind that it is the mBDNF and not the proBDNF that causes LTP, the proform of BDNF actually has a higher affinity for p75NTR and promotes LTD.

In Drosophila, mutant huntington protein has been shown to cause neurodegeneration, but its role in learning and memory has remained unexplored (Steffan et al, 2001; Taylor et al, 2003). However, the neurodegenerative phenotype caused by expression of the mutant huntington protein has been demonstrated to be lessoned by HDAC inhibition and by CBP overexpression (Steffan et al, 2001; Taylor et al, 2003). Both HDAC inhibition and CBP overexpression should in theory promote an increase CREB mediated gene transcription. We demonstrated that expressing mutant huntington in the MBs of Drosophila resulted in short-term memory deficits in 5 day old flies, whereas expression of the wt huntington protein in the MBs did not impair short-term memory (data not shown). Since we knew that we could use a PDE-4 inhibitor or mGluR antagonists or lithium to increase cAMP levels thereby promoting CREB mediated transcription, we decided to see if these compounds could restore the memory deficit in Drosophila expressing mutant huntington in the MBs. The mGluR antagonist LY341495 restored short-term memory in Drosophila expressing mutant huntington protein in the MBs, p<0.05.

TABLE 3
Drosophila treated with LY341495.

| | Naive | s* | (n) | STM** | s | (n) |
|---|---|---|---|---|---|---|
| 93 HD × 30y | 60.8 | 3.8 | (19) | 60.0 | 5.2 | (18) |
| 93 HD × 30y | 75.1 | 3.5 | (20) | 65.8 | 4.0 | (19) |

*s = standard error
**STM = 60 minutes post training.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1: A method of evaluating a compound for the ability to reduce a mental defect in a metazoan, where the mental defect is caused by a disease, wherein the disease is Fragile X syndrome, a tauopathy, Huntington's disease, neurofibromatosis 1, Parkinson's disease, or a disease analogous in the metazoan to Fragile X syndrome, a tauopathy, Huntington's disease, neurofibromatosis 1, or Parkinson's disease, the method comprising determining whether the compound reduces a mental effect of an analogous disease in a Drosophila.

2: The method of claim 1, wherein the mental defect is in memory, orientation, learning, attention, reasoning, language, and/or the ability to perform simple tasks.

3: The method of claim 1, wherein the disease is Fragile X syndrome and the analogous disease in a Drosophila is caused by a deficiency in a dFMR1 protein.

4: The method of claim 1, wherein the disease is a tauopathy and the analogous disease in a Drosophila is caused by expression of a human tau protein.

5-6. (canceled)

7: The method of claim 1, wherein the disease is Alzheimer's and the analogous disease in a Drosophila is caused by expression of a mutant presenilin gene associated with Alzheimer's.

8-11. (canceled)

12: The method of claim 1, wherein the compound is an inhibitor of a glutamate receptor (GluR).

13-34. (canceled)

35: A method of improving learning or memory in a mammal, the method comprising treating the mammal with a compound in an amount sufficient to improve learning or memory in the mammal, wherein the compound inhibits expression or activity of a group II or group III metabotropic glutamate receptor (mGluR), an inositol trisphosphate receptor (InsP3R), a glycogen synthase kinase-3β (GSK-3β), or a phosphodiesterase-4 (PDE-4) in the mammal.

36: The method of claim 35, wherein the mammal has Fragile X syndrome, a tauopathy, Huntington's disease, neurofibromatosis 1, Parkinson's disease, or a non-human disease analogous to Fragile X syndrome, a tauopathy, Huntington's disease, neurofibromatosis 1, or Parkinson's disease.

37: The method of claim 35, wherein the mammal is treated with LiCl.

38: The method of claim 35, wherein the mammal is treated with an inhibitor of a group II mGluR where the inhibitor is at a concentration that it is specific for a group II mGluR.

39: The method of claim 35, wherein the mammal is treated with an inhibitor of a group III mGluR where the inhibitor is at a concentration that is specific for a group III mGluR.

40: The method of claim 35, wherein the compound is 2-methyl-6-(phenylethynyl)pyridine (MPEP), 2-amino-4-phosphonobutanoic acid (AP-4), (RS)-α-methylserine-O-phosphate monophenyl ester, (RS)-1-amino-5-phosphonoindan-1-carboxylic acid [(RS)-APICA], (RS)-α-methyl-4-tetrazolylphenylglycine (MTPG), (2S)-α-ethylglutamic acid (EGLU), (2S)-2-amino-2-[(1S,2S)-2-carboxycycloprop-1-yl]-3-(xanth-9-yl) propionic acid (LY341495), TDZD-8,1-azakenpaullone, MAP4, or 4-[3-(Cyclopentyl)-4-methoxyphenyl]-2-pyrrolidinone (rolipram).

41: The method of claim 35, wherein the compound comprises a nucleic acid.

42-44. (canceled)

45: A method of treating a mammal having Fragile X disease or a non-human disease analogous to Fragile X disease, the method comprising treating the mammal with a compound that inhibits expression or activity of a group II or group III metabotropic glutamate receptor (mGluR), an inositol trisphosphate receptor (InsP3R), a glycogen synthase kinase-3β (GSK-3β), or a phosphodiesterase-4 (PDE-4) in the mammal.

46: The method of claim 45, wherein the mammal exhibits a deficiency in memory, orientation, learning, attention, reasoning, language and/or the ability to perform simple tasks.

47: The method of claim 45, wherein the mammal is treated with LiCl.

48: The method of claim 45, wherein the mammal is treated with an inhibitor of a group II mGluR where the inhibitor is at a concentration that it is specific for a group II mGluR.

49: The method of claim 45, wherein the mammal is treated with an inhibitor of a group III mGluR where the inhibitor is at a concentration that is specific for a group III mGluR.

50: The method of claim 45, wherein the compound is 2-methyl-6-(phenylethynyl)pyridine (MPEP), 2-amino-4-phosphonobutanoic acid (AP-4), (RS)-α-methylserine-O-phosphate monophenyl ester, (RS)-1-amino-5-phosphonoindan-1-carboxylic acid [(RS)-APICA], (RS)-α-methyl-4-tetrazolylphenylglycine (MTPG), (2S)-α-ethylglutamic acid (EGLU), (2S)-2-amino-2-[(1S,2S)-2-carboxycycloprop-1-yl]-3-(xanth-9-yl) propionic acid (LY341495), TDZD-8,1-azakenpaullone, MAP4, or 4-[3-(Cyclopentyl)-4-methoxyphenyl]-2-pyrrolidinone (rolipram).

51: The method of claim 45, wherein the compound comprises a nucleic acid.

52-88. (canceled)
